(19) United States
(12) Patent Application Publication
(10) Pub. No.: US 2021/0322417 A1
(43) Pub. Date: Oct. 21, 2021

(54) GASTRIC CANCER TREATMENTS

(71) Applicant: The Jackson Laboratory, Bar Harbor, ME (US)

(72) Inventors: Chengsheng Zhang, Bar Harbor, ME (US); Charles Lee, Bar Harbor, ME (US); Gang Ning, Bar Harbor, ME (US); Yun-Suhk Suh, Bar Harbor, ME (US); Qihui Zhu, Bar Harbor, ME (US)

(73) Assignee: The Jackson Laboratory, Bar Harbor, ME (US)

(21) Appl. No.: 17/245,240

(22) Filed: Apr. 30, 2021

Related U.S. Application Data
(63) Continuation of application No. PCT/US2019/059306, filed on Nov. 1, 2019.
(60) Provisional application No. 62/754,504, filed on Nov. 1, 2018.

(51) Int. Cl. A61K 31/517; A61K 31/519; A61K 31/436; A61K 31/5377; A61P 35/00

(52) U.S. Cl. CPC A61K 31/517; A61K 31/519; A61P 35/00; A61K 31/5377; A61K 31/436

(57) ABSTRACT

Provided herein, in some embodiments, are methods, compositions, and kits for treating lapatinib-resistant gastric cancer.

### RELATED APPLICATION

This application claims the benefit under 35 U.S.C. § 119(e) of U.S. provisional application No. 62/754,504, filed Nov. 1, 2018, which is incorporated by reference herein in its entirety.

### BACKGROUND

Gastric cancer (C) is one of the leading causes of deaths of all malignancies worldwide¹⁻². According to American Cancer Society's estimation, there are about 26.240 new GC cases and 10.800 deaths in US in 2017³. As an oncogenic driver, human epidermal growth factor receptor 2 (HER2) gene amplification or oncoprotein overexpression occurs in approximately 13-23% of GC cases⁴. It has been reported that HER2 amplification and overexpression was associated with poor prognosis in GC patients⁵⁻⁶.

HER2 is transactivated through heterodimerization with other HER family members. Notably, HER2 overexpression promotes tumor cell proliferation, adhesion, migration and survival by constitutive activation of cascades in the downstream signaling transduction of the Ras/Raf/Mitogen activated protein kinase (MAPK) and phosphatidylinositol 3 kinase (PI3K)/AKT/Mammalian target of rapamycin (mTOR) pathways⁷. HER2 targeted therapy and its efficacy have been achieved with monoclonal antibody trastuzumab (HERCEPTIN®) and small molecule tyrosine kinase inhibitor lapatinib (TYKERB®) in breast cancer⁸. However, the clinical trial data of HER2-amplified GC shown that trastuzumab only improved overall survival for 2.7 months whereas lapatinib failed to improve survival in HER2-positive GC patients⁹⁻¹¹.

### SUMMARY

In order to understand the molecular mechanisms involved in lapatinib resistance in HER2 amplified GC cells, we employed an unbiased, genome-scale screening approach with the pooled CRISPR library to identify genes that may be associated with resistance to lapatinib. We found that loss of function mutations in C-Terminal Src Kinase (CSK) or Phosphatase and Tensin Homolog (PTEN) conferred lapatinib resistance in human HER2 amplified GC cell lines NCI-N87 and OE19, respectively. Moreover, we observed the hyperactivation of PI3K and MAPK signaling pathways in CSK or PTEN null cells and the resistance could be overcome by combinational treatment of the cells with lapatinib. PI3K inhibitor copanlisib (ALIQOPA™) and mitogen-activated protein kinase (MEK) inhibitor trametinib (MEKINIST®), suggesting that these signaling pathways may play important roles in lapatinib resistance. This study provides insights for understanding the resistant mechanism of HER2 targeted therapy and novel strategies that may ultimately overcome resistance or limited efficacy of anti-HER2 axis treatments for GC.

The present disclosure provides methods of treating gastric cancer in a subject, the methods comprising: (a) administering to the subject lapatinib; and (b) administering to the subject a phosphoinositide 3-kinase (PI3K) inhibitor, a MEK inhibitor, or a combination of a PI3K inhibitor and a MEK inhibitor.

In some embodiments, the gastric cancer is HER2-amplified gastric cancer.

In some embodiments, step (b) comprises administering to the subject a PI3K inhibitor and a MEK inhibitor. In some embodiments, the PI3K inhibitor is copanlisib. In some embodiments, the MEK inhibitor is trametinib.

In some embodiments, the lapatinib and the PI3K inhibitor, the lapatinib and the MEK inhibitor, or the lapatinib, the PI3K inhibitor, and the MEK inhibitor are administered simultaneously.

In some embodiments, the ratio of lapatinib to PI3K inhibitor is 1:2, the ratio of lapatinib to MEK inhibitor is 1:2, and the ratio of PI3K inhibitor to MEK inhibitor is 1:1.

In some embodiments, the lapatinib, the PI3K inhibitor, or the MEK inhibitor is administered intravenously or orally.

In some embodiments, lapatinib, copanlisib, and trametinib are administered in amounts effective to reduce the volume of a gastric tumor in a subject by at least 70% (e.g., by at least 75%, at least 80%, at least 85%, at least 90%, or at least 95%).

In some embodiments, the gastric cancer cells do not express, or express a reduced level of, a CSK gene and/or a PTEN gene.

The present disclosure also provides methods comprising (a) contacting gastric cancer cells with lapatinib; and (b) contacting the gastric cancer cells with a PI3K pathway inhibitor, a MAPK pathway inhibitor, a SRC family inhibitor, an mTOR inhibitor, or a combination thereof.

In some embodiments, the gastric cancer cells are HER2-amplified gastric cancer cells.

In some embodiments, the SRC family inhibitor comprises saracatinib. In some embodiments, the mTOR inhibitor comprises rapamycin. In some embodiments, the PI3K pathway inhibitor is a PI3K inhibitor. In some embodiment, the PI3K inhibitor is copanlisib or LY294002. In some embodiments, the MAPK pathway inhibitor comprises a MEK inhibitor. In some embodiments, the MEK inhibitor comprises trametinib.

In some embodiments, the gastric cancer cells do not express, or express a reduced level of, a gene selected from CSK, PTEN, BAX, KCTD5, KEAP1, NF1, and TADA1.

The present disclosure also provides kits comprising: (a) lapatinib; and (b) a PI3K pathway inhibitor, a MAPK pathway inhibitor, or a PI3K pathway inhibitor and a MAPK pathway inhibitor. In some embodiments, the PI3K pathway inhibitor is a PI3K inhibitor. In some embodiments, the PI3K inhibitor is capanlisib. In some embodiments, the MAPK pathway inhibitor comprises a MEK inhibitor. In some embodiments, the MEK inhibitor comprises trametinib. In some embodiments, the kit comprises lapatinib, copanlisib, and trametinib.

Also provided herein are methods comprising: (a) delivering in vitro to control cells and to human gastric cancer cells harboring HER2 amplification a pooled genome-scale CRISPR-Cas9 knockout library; (b) treating the controls cells and the human gastric cancer cells of step (a) with lapatinib; (c) extracting DNA from the lapatinib-treated controls cells and the lapatinib-treated human gastric cancer cells of step (b); (d) sequencing the DNA extracted from step (c); and (e) identifying from the sequenced DNA of step (d) candidate loss-of-function genes that may contribute to lapatinib resistance.

In some embodiments, a pooled genome-scale CRISPR-Cas9 knockout library is delivered using a lentiviral delivery system.

In some embodiments, the method further comprises step (f) validating at least one of the candidate loss-of-function genes. In some embodiments, validating comprises delivering In vitro to control cells and to human gastric cancer cells a gRNA, treating the human gastric cancer cells with lapatinib, and assessing cell viability to evaluate lapatinib resistance. In some embodiments, cell viability is assessed in step (f) by measuring caspase-3/7 activation in the lapatinib-treated control cells and the lapatinib-treated human gastric cancer cells.

### BRIEF DESCRIPTION OF THE DRAWINGS

FIGS. 1A-1C: Genome-sale CRISPR library knockout screening for genes associated with Lapatinib resistance in GC cell lines. (FIG. 1A) Schematic diagram of the CRISPR library screening strategy. The loss of function screening was performed with the infection of pooled lentivirus containing the GeCKO library V2.0 on N87 and OE19 cells, followed by puromycin selection and Lapatinib treatment. The cells were harvested for genomic DNA to PCR the gRNAs and the subsequent sequencing after 14 days post treatment. (FIG. 1B) The distribution of gRNA frequencies in the untreated (baseline), the vehicle-treated (DMSO), and Lapatinib-treated N87 and OE19 cells, respectively. (FIG. 1C) Scatterplot showing identification of top 10 candidate genes using Model-based Analysis of Genome-wide CRISPR-Cas9 Knockout (MAGeCK).

FIGS. 2A-2E. Functional validation study of CSK or PTEN null N87 and OE19 cells. (FIG. 2A) Cell viability of CSK or PTEN knockout OE19 cells treated with indicated doses of Lapatinib. OE19 cells were transduced with lentiviruses carrying gRNAs targeting CSK, PTEN, or non-targeting control gRNA. The drug resistance of the cells from each group was measured by calculating the relative percentage of cell viability. CSK or PTEN protein expressions were evaluated by Western blotting. (FIG. 2B) Cell viability of CSK or PTEN knockout N87 cells treated with different doses of Lapatinib. N87 cells transduced with non-targeting gRNA as control. Shift between control cells and gene knockout cells in the dose response curve displays the reduced sensitivity to Lapatinib in the GC cell lines. CSK or PTEN protein expressions were evaluated by Western blotting. (FIG. 2C) Caspase-Glo 3/7 assay analysis to examine Lapatinib induced caspase-3/7 activity after 48 h treatment in CSK or PTEN null cells OE19 and N87 cells. OE19 or N87 cells transduced with virus carrying non-targeting gRNA as control. (FIG. 2D) Cell viability curve of KEAP1, BAX, MED24 or TADA1 knockout OE19 cells treated with indicated doses of Lapatinib, respectively. OE19 cells were transduced with lentivirus carrying gRNAs targeting the indicated gene individually. The drug resistance of gene knockout and the control cells were measured by the relative percentage of cell viability. (FIG. 2E) Cell viability curve of KCTD5 or NF1 knockout OE19 cells treated with indicated doses of Lapatinib, respectively. OE19 cells were transduced with lentivirus carrying gRNAs targeting the indicated gene individually. The drug resistance of gene knockout and the control cells were examined by the relative percentage of cell viability.

FIGS. 3A-3F: Protein interaction network prediction, gene expression profile and pathway analysis of CSK or PTEN knockout cell lines. Protein interaction network (FIG. 3A) and the predicted partners (FIG. 3B) were analyzed by STRING. CSK. PTEN and HER2(ERBB2) were mapped by searching the STRING (Search Tool for the Retrieval of Interacting Genes/Proteins) database version 10.5. In the resulting protein association network, proteins are presented as nodes which are connected by lines whose thickness represents the confidence level. (FIG. 3C) Heatmap of 139 DEGs between CSK null cells vs parental N87 cells (Fold change >1.5. FDR<0.1). (FIG. 3D) Heatmap of 997 DEGs between PTEN null cells vs parental OE19 cells (Fold change >1.5 FDR<0.1). (FIG. 3E) The bar plot depicts the enriched pathway among the DEGs between CSK null cells (N87-CSK-gRNA) and parental N87 cells (N87) by KEGG pathway analysis. (FIG. 3F) The bar plot depicts the enriched pathway among the DEGs between PTEN null cells (OE19-PTEN-gRNA) and parental OE19 cells (OE19) by KEGG pathway analysis.

FIGS. 4A-4D: Up-regulation of PI3K/AKT and MAPK pathways in the CSK or PTEN knockout GC cells. (FIG. 4A) The levels of phosphorylated and total proteins of AKT and MAPK were assessed by Western blotting in OE19 cells transduced with lentivirus carrying CSK targeting gRNAs or PTEN targeting gRNAs, respectively. OE19 cells transduced with non-targeting gRNA as control. (FIG. 4B) PTEN aid CSK protein expression was examined by Western blotting in CSK knockout OE19 cell lines and PTEN knockout OE19 cell lines, respectively. OE19 cells transduced with non-targeting gRNA as control. (FIG. 4C) The levels of phosphorylated and total proteins of AKT and MAPK were assessed by Western blots in N87 cells transduced with CSK targeting gRNAs or PTEN targeting gRNAs, respectively. N87 cells transduced with non-targeting gRNA as control. (FIG. 4D) PTEN protein and CSK protein expression was examined by Western blotting in CSK knockout N87 cell lines and PTEN knockout N87 cell lines, respectively. N87 cells transduced with non-targeting gRNA as control.

FIGS. 5A-5F: Pharmacological inhibition of PI3K, MAPK and SRC signaling pathway re-sensitizes resistant GC cells to Lapatinib. OE19 cells transduced with CSK targeting gRNAs or PTEN targeting gRNAs were used for following test. OE19 cells transduced with non-targeting gRNA as control. (FIG. 5A) Growth curve of test groups with 0.05 μM Lapatinib in combination with indicated dose of trastuzumab for 6 days. (FIG. 5B) Growth curve of test groups treated with indicated dose of SRC inhibitor AZD0530 for 6 days. (FIG. 5C) Growth curve of test group treated with 0.05 μM Lapatinib in combination with indicated dose of PI3K inhibitor Copanlisib for 6 days. (FIG. 5D) Growth curve of test groups treated with 0.05 μM μLapatinib in combination with different doses of mTOR inhibitor Rapamycin for 6 days. (FIG. 5E) Growth curve of test groups treated with 0.05 μM Lapatinib in combination with different doses of MEK inhibitor Trametinib for 6 days. (FIG. 5F) Inhibition effect of 0.05 μM Lapatinib alone or in combination with 0.1 μM trametinib or/and 0.1 μM copanlisib for 6 days.

FIGS. 6A-6D: (FIG. 6A) Growth curve of test groups of N87 cells with 0.01 μM Lapatinib in combination with indicated doses of Trastuzumab for 6 days. (FIG. 6B) Pharmacological inhibition of PI3K, MAPK signaling pathway re-sensitizes CSK or PTEN null GC cells to Lapatinib. Inhibition effect of 0.01 μM Lapatinib alone or in combination with 0.1 μM Trametinib or/and 0.1 μM Copanlisib for 6 days. N87 cells transduced with CSK targeting gRNAs or PTEN targeting gRNAs were used for the test. N87 cells transduced with non-targeting gRNA as control. (FIG. 6C) Growth curve of CSK and PTEN gene knockout OE19 cell lines treated with 0.05 μM Lapatinib in combination with different doses of PI3K inhibitor LY294002 for 6 days. OE19 cells transduced with non-targeting gRNA as control. (FIG. 6D) Pharmacological inhibition of PI3K, MAPK signaling pathway re-sensitizes NF1 or KEAP1 null GC cells to Lapatinib. Inhibition effect of 0.05 μM Lapatinib alone or in combination with 0.1 μM Trametinib or/and 0.1 μM Copanlisib for 6 days. OE19 cells transduced with NF1 targeting gRNAs or KEAP1 targeting gRNAs were used for the test. OE19 cells transduced with non-targeting gRNA as control.

FIG. 7: A schematic diagram showing potential HER2-related signaling pathways and action mechanisms of various inhibitors in HER2 amplified GC. Heterodimerization of HER2 with other HER family members (EGFR, HER3, HER4) result in tyrosine kinase activation with the subsequent signaling cascade, including members of MAPK and PI3K/AKT/mTOR pathways. As a result of these signaling pathways activation, different nuclear factors are recruited and modulate the transcription of different genes involved in cell-cycle progression, proliferation, and survival. Trastuzumab inhibits HER2 by targeting its extracellular domain, whereas Lapatinib inhibits both HER2 and EGFR by inhibiting the intracellular tyrosine kinases. HER2 targeted therapy could be interrupted by re-activation MAPK and PI3K/AKT/mTOR pathways by compensatory activation of MET, IGF-RI. HER3 or loss of function mutations of tumor suppressors genes such as CSK, PTEN, NF1. In addition, drug resistance could be conferred by loss of function mutations of downstream genes such as KEAP1 and BAX by dysregulation of cellular antioxidants, xenobiotic detoxification enzymes and apoptosis, respectively. Lapatinib combining with SRC inhibitor AZD0530, PI3K inhibitor Copanlisib, mTOR inhibitor Rapamycin, or MEK inhibitor Trametinib could counteract the resistance at different level, respectively. A combinational treatment strategy with Lapatinib, Copanlisib and Trametinib is demonstrated more effective for HER2 amplified GC with CSK. PTEN, NF1and KEAP1 mutations in this study.

FIG. 8: Graphs of data showing that compared with N87-WT tumors, N87-CSK^{-/-} tumors arc relatively insensitive to lapatinib, and N87-PTEN^{-/-} tumors are resistant to lapatinib treatment.

FIG. 9: Schematic depicting an experiment designed to test the efficacy of lapatinib+trametinib+copanlisib with other treatment conditions, including gastric cancer standard chemotherapy agent fluorouracil.

FIGS. 10A-10B: Graphs of data from the in vivo test with the N87-PTEN^{/} xenograft tumor model, where a significant effect upon tumor growth was observed with the combination of lapatinib, trametinib and copanlisib (2-way ANOVA ***. P<0.0001) when compared with vehicle, lapatinib alone, or 5-FU treatment groups, respectively. Similarly. when the mass of the tumors at endpoint were compared, the three drug combinations showed significant improvement over lapatinib alone (Unpaired t test: ***. P=0.0008) and 5-FU (Unpaired t test: **, P=0.0013) Error bars. SD (FIG. 10A). Similar result was obtained from the experiment with N87-CSK−/− xenograft (FIG. 10B), although N87-CSK^{-/-} tumors seem less resistant to lapatinib treatment than N87-PTEN^{-/-}.

### DETAILED DESCRIPTION

Clinical trial data of HER2-amplified gastric cancer (GC) shown that trastuzumab only improved overall survival for 2.7 months whereas lapatinib failed to improve survival in HER2-positive GC patients⁹⁻¹¹. The unsatisfactory results may be attributed to the intrinsic or acquired resistance to HER2-targeted therapy. To improve the efficacy of HER2 targeted therapy in GC patients, there is an urgent need to elucidate the mechanisms of resistance. Previous studies suggested that the MET and CCNE1 amplifications were involved in lapatinib resistance as compensation for HER family inhibition by re-stimulating downstream signaling pathways¹²⁻¹³, but the underlying molecular mechanisms remain largely unknown.

CRISPR-Cas9 gene editing-based library screening has been proved to be a very efficient tool to screen gene mutations that confer drug resistance in cell-based assays¹⁴. It is considered superior to shRNA library screening because of its robustness, higher specificity and efficiency¹⁵⁻¹⁶. To identify genes that may be associated with Lapatinib resistance, in this study, we employed a gene knockout screening approach with a pooled genome-scale CRISPR-Cas9 knockout (GeCKO) V2 library, targeting 19.050 genes with 123,411 single guide RNAs (gRNAs) (6 gRNAs per gene) on two HER2 amplified GC cell lines, NCI-N87(N87) and OE19, respectively. We identified and validated a set of genes whose loss of function mutations contribute to Lapatinib resistance, including CSK, PTEN, BAX, NF1, KEAP1, TADA1, and KCTD5. Further studies on the CSK or PTEN null GC cells demonstrated that deletion of these two genes conferred resistance by restoring the PI3K/AKT and MAPK pathways. Moreover, the resistance could be overcome by combinational treatment of the cells with Lapatinib, PI3K inhibitor Copanlisib and MEK inhibitor Trametinib. Our findings not only reveal the genes and signal pathways that contribute to Lapatinib resistance, but also provide a potential treatment strategy for a subset of HER2-amplified GC.

Some aspects of the present disclosure provide methods of treating gastric cancer in a subject that include administering to the subject lapatinib and a PI3K pathway inhibitor, a MAPK pathway inhibitor, a SRC family inhibitor, an mTOR inhibitor, or a combination thereof.

As used herein, administering refers to delivering to a cell or subject in need thereof a an agent (e.g., lapatinib, a PI3K inhibitor, and/or a MEK inhibitor). Non-limiting examples of routes of administration include: oral (e.g. tablet, capsule), intravenous, subcutaneous, inhalation, intranasal, intrathecal, intracerebral, intramuscular, intraarterial, and intraneural.

In some embodiments, the present disclosure provides methods for treating a subject who has or is suspected of having gastric cancer (stomach cancer). Gastric cancer is one of the most common cancers worldwide, with approximately 25,000 new patients diagnosed annually in the United States. Most (˜95%) of gastric cancers are adenocarcinomas which develop from the mucosal cells lining the stomach. Lymphomas derived from the immune system, gastrointestinal stromal tumors derived from interstitial cells in the stomach wall, and carcinoid tumors derived from endocrine cells in the stomach also occur. Signs and symptoms of gastric cancer may include: fatigue, feeling bloated after eating, feeling full after eating small amounts of food, severe and persistent heartburn, severe and constant indigestion, unexplained and persistent nausea, stomach pain, persistent vomiting, and unintentional weight loss.

Treatment for gastric cancer includes surgery to resect the cancerous portion of the stomach, radiation therapy, and drugs FDA-approved in the US for treating gastric cancer. Non-limiting examples of these drugs include: ramuerirumab (CYRAMZA®), docetaxel (TAXOTERE®), doxorubicin hydrochloride, fluorouracil (also referred to as 5-FU), mitomycin C, pembrolizumab (KEYTRUDA®), ramucirumab (CYRAMZA®), and trastuzumab (HERCEPTIN®).

A key prognostic indicator in gastric cancer is the level of human epidermal growth factor 2 (HER2) expression, wherein amplification of HER2 expression (HER2-amplified) is associated with decreased survival, more aggressive cancer proliferation, and higher frequencies of HER2-positive tumors compared with non-HER2 amplified gastric cancers. Although HER2-amplified breast cancers respond to treatment with either the HER2 inhibitors lapatinib or trastuzumab, patients with HER2-amplified gastric cancer do not, suggesting that there are genes other than HER2 which are differentially regulated in gastric cancer relative to breast cancer which promote resistance to lapatinib and trastuzumab.

HER2 is a membrane receptor tyrosine kinase the amplification or over-expression of which has been shown to play an important role in the development and progression of certain aggressive breast, gastric, ovarian, uterine, and lung cancers. The gene which encodes HER2, ERBB2, is therefore identified as an oncogene. Upon extracellular ligand binding, HER2 autophosphorylates tyrosine residues in its intracellular domain and activates numerous pathways which promote cell proliferation and inhibit apoptosis, including mitogen-activated protein kinase (MAPK), phosphoinositide 3-kinase (PI3K/Akt), phospholipase C, protein kinase C (PKC), and signal transducer and activator of transcription (STAT) pathways.

### Lapatinib

Lapatinib is a HER2 (HER2/ERBB2) and epidermal growth factor receptor (EGFR/ERBB1/HER1) tyrosine kinase inhibitor. Lapatinib passes through the plasma membrane and binds to the intracellular tyrosine kinase phosphorylation domain on the HER2 and EGFR receptors to prevent receptor autophosphorylation upon ligand binding, inhibiting HER2 receptor and EGFR receptor activation of downstream signaling pathways. Lapatinib is FDA-approved in the US for treating HER2+ metastatic breast cancer. It is administered orally in combination with the chemotherapeutic agent capecitabine.

In some embodiments, lapatinib is administered at a dose of 50 mg/kg to 200 mg/kg. For example, lapatinib may be administered at a dose of 50-175 mg/kg, 50-150 mg/kg, 50-125 mg/kg, 50-100 mg/kg, 50-75 mg/kg, 750-200 mg/kg, 750-175 mg/kg, 750-150 mg/kg, 750-125 mg/kg, 750-100 mg/kg, 100-200 mg/kg, 100-175 mg/kg, 100-150 mg/kg, 100-125 mg/kg, 125-200 mg/kg, 125-175 mg/kg, 125.150 mg/kg, 150-200 mg/kg, 150-175 mg/kg, or 175-200 mg/kg. In some embodiments, lapatinib is administered at a dose of 50 mg/kg, 75 mg/kg, 100 mg/kg, 125 mg/kg, 150 mg/kg, 175 mg/kg, or 200 mg/kg.

In some embodiments, lapatinib is administered at a dose of 500 mg to 2000 mg. For example, lapatinib may be administered at a dose of 500-1750 mg, 500-1500 mg, 500-1250 mg, 500-1000 mg, 500-750 mg, 750-2000 mg, 750-1750 mg, 750-1500 mg, 750-1250 mg, 750-1000 mg, 1000-2000 mg, 1000-1750 mg, 1000-1500 mg, 1000-1250 mg, 1250-2000 mg, 1250-1750 mg, 1250-1500 mg, 1500-2000 mg, 1500-1750 mg, or 1750-2000 mg. In some embodiments, lapatinib is administered at a dose of 500 mg, 750 mg, 1000 mg, 1250 mg, 1500 mg, 1750 mg, or 2000 mg.

In some embodiments, a dose of lapatinib is administered as an oral tablet. Other routes of administration, as described below, may be used.

In some embodiments, a dose of lapatinib is administered once a day, twice a day, or three times a day, for example, over the course of 10 days, 20 days, 30 days, 60 days, 90 days, 120 days, 150 days, or longer.

In some embodiments, a 1250 mg dose of lapatinib is administered as an oral tablet (or as five 250 mg oral tablets) once daily on a 21-day cycle.

### PI3K Pathway Inhibitors

Phosphoinositide 3-kinases (PI3Ks) are a family of intracellular lipid kinases that produce phospholipids in response to signals from various growth factors and cytokines.

These phospholipids then activate the serine/threonine kinase AKT and other downstream effector pathways that promote cell growth, proliferation, and survival. PI3K enzymes are divided into three classes based on structural characteristics and substrate specificity. Class I enzymes are the most well-characterized, include multiple subunits, and are activated by cell surface receptors. Class II enzymes include a single subunit and are activated by cell surface receptors and transmembrane proteins. Class III enzymes include a single subunit and are thought to function as nutrient-regulated lipid kinases. The activity of the PI3K pathway is upregulated in some cancer cells compared with non-cancer cells. A PI3K inhibitor is an agent that disrupts the action of at least one class of PI3K enzymes. An agent is a compound or drug that is administered to a cell or subject in need thereof. Non-limiting examples of PI3K inhibitors include: copanlisib (Class I), LY294002 (Class I), taselisib (Class I), idelalisib (Class I), buparlisib, duvelisib, alpelisib, umbralisib, PX-466, dactolisib, CUDC-907,v, ME-401, IPI-549, SF1126, PR6530, INK1117, pictilisib, XL147, palomid 529, GSK1059615, ZSTK474, PWT33597, IC87114, TG100-115, CAL263, RP6503, PI-103, GNE-477, and AEZS-136.

In some embodiments, a PI3K inhibitor comprises copanlisib. Copanlisib (ALIQOPA™) is a PI3K Class I enzyme inhibitor that selectively and simultaneously binds two subunits of the Class I PI3K enzyme, inhibiting downstream signaling activity which promotes cell proliferation and survival. Copanlisib is FDA-approved in the US for the treatment of relapsed follicular lymphoma. In some embodiments, copanlisib is administrated intravenously by infusion.

In some embodiments, a PI3K inhibitor is LY294002. LY294002 is a morpholine-containing compound which binds and partially blocks the ATP-binding site of PI3K kinase enzymes. LY294002 inhibits the growth of ovarian carcinoma in vitro and in vivo.

In some embodiments, a PI3K inhibitor (e.g., copanlisib) is administered at a dose of 1 mg/kg to 10 mg/kg. For example, a PI3K inhibitor may be administered at a dose of 1-9 mg/kg, 1-8 mg/kg, 1-7 mg/kg, 1-6 mg/kg, 1-5 mg/kg, 1-4 mg/kg, 1-3 mg/kg, 1-2 mg/kg, 2-10 mg/kg, 2-9 mg/kg, 2-8 mg/kg, 2-7 mg/kg, 2-6 mg/kg, 2-5 mg/kg, 2-4 mg/kg, 2-3 mg/kg, 3-10 mg/kg, 3-9 mg/kg, 3-8 mg/kg, 3-7 mg/kg, 3-6 mg/kg, 3-5 mg/kg, 3-4 mg/kg, 4-10 mg/kg, 4-9 mg/kg, 4-8 mg/kg, 4-7 mg/kg, 4-6 mg/kg, 4-5 mg/kg, 5-10 mg/kg, 5-9 mg/kg, 5-8 mg/kg, 5-7 mg/kg, 5-6 mg/kg, 6-10 mg/kg, 6-9 mg/kg, 6-8 mg/kg, 6-7 mg/kg, 7-10 mg/kg, 7-9 mg/kg, 7-8 mg/kg, 8-10 mg/kg, 8-9 mg/kg, 8-7 mg/kg, or 9-10 mg/kg. In some embodiments, a PI3K inhibitor is administered at a dose of 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg.

In some embodiments, a PI3K inhibitor (e.g., copanlisib) is administered at a dose of 10 mg to 100 mg. For example, a PI3K inhibitor may be administered at a dose of 10-90 mg, 10-80 mg, 10-70 mg, 10-60 mg, 10-50 mg, 10-40 mg, 10-30 mg, 10-20 mg, 20-100 mg, 20-90 mg, 20-80 mg, 20-70 mg, 20-60 mg, 20-50 mg, 20-40 mg, 20-30 mg, 30-100 mg, 30-90 mg, 30-80 mg, 30-70 mg, 30-60 mg, 30-50 mg, 30-40 mg, 40-100 mg, 40-90 mg, 40-80 mg, 40-70 mg, 40-60 mg, 40-50 mg, 50-100 mg, 50-90 mg, 50-80 mg, 50-70 mg, 50-60 mg, 60-100 mg, 60-90 mg, 60-80 mg, 60-70 mg, 70-100 mg, 70-90 mg, 70-80 mg, 80-100 mg, 80-90 mg, 80-70 mg, or 90-100 mg. In some embodiments, a PI3K inhibitor is administered at a dose of 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg.

In some embodiments, a dose of a PI3K inhibitor (e.g., copanlisib) is administered as an intravenous (IV) infusion. Other routes of administration, as described below, may be used.

In some embodiments, a dose of a PI3K inhibitor (e.g., copanlisib) is administered once a day, twice a day, or three times a day, for example, over the course of 10 days, 20 days, 30 days, 60 days, 90 days, 120 days, 150 days, or longer.

In some embodiments, a 60 mg dose of a PI3K inhibitor (e.g., copanlisib) is administered as an IV infusion over 1 hour on Day 1, 8, 15 of a 28-day cycle on an intermittent schedule (3 weeks on, 1 week off).

### MAPK Pathway Inhibitors

In numerous cancers, including melanoma and non-Hodgkin's lymphoma, a defect in the mitogen-activated protein kinase (MAPK) pathway leads to uncontrolled growth. The MAPK pathway comprises a variety of a highly conserved serine/threonine kinase enzymes involved in critical cellular processes such as proliferation, differentiation, apoptosis, and survival. The MAPK pathway is activated by growth factors and cytokines that bind to and activate the transmembrane receptor tyrosine kinases ARAF, BRAF, or CRAF. These activated kinases then phosphorylate the MAPK/ERK kinases (MEK1/2), which phosphorylate and activate the mitogen-activated protein kinases (MAPKs) ERK1/2, which translocate to the nucleus and promote the transcription of genes involved in cell proliferation, differentiation, migration, and apoptosis. A MAPK pathway inhibitor is an agent which selectively down-regulates the activation or activity of at least one enzyme in the MAPK pathway. Non-limiting examples of MAPK pathway inhibitors include: trametinib, sorafenib, SB590885, PLX4720, XL281, RAF265, encorafenib, dabrafenib, vemurafenib, cobimetinib, CI-1040, PD0325901, binimetinib, and selumetinib.

The activity of the MEK pathway, in particular, is upregulated in some cancer cells compared to non-cancerous cells. MEK phosphorylate and activate mitogen-activated protein kinases such as ERK. ERK then phosphorylates and regulates the activities of numerous transcription factors, including C-myc.

In some embodiments, the MAPK pathway inhibitor comprises a MEK inhibitor. A MEK inhibitor is an agent that disrupts the activity of a MEK1 and/or MEK2 enzyme. These inhibitors block either the activation of MEK1/MEK2 or the downstream phosphorylation of the MEK1/2 targets EKR1/2. Non-limiting examples of MEK inhibitors include: trametinib, cobimetinib, binimetinib, selumetinib. PD-325901. CI-1040, and TAK-733.

In some embodiments, the MEK inhibitor comprises trametinib. Trametinib is an adenosine-triphosphate-noncompetitive inhibition of both activation and kinase activity of MEK1 and MEK2. Binding of trametinib inhibits the phosphorylation of MEK1/2, leading to decreased kinase activity In some embodiments, trametinib is administered orally. Trametinib is FDA-approved in the US for the treatment of melanoma, non-small cell Jung cancer, and thyroid cancer.

In some embodiments, a MEK inhibitor (e.g., trametinib) is administered at a dose of 0.3 mg/kg to 1 mg/kg. For example, a MEK inhibitor may be administered at a dose of 0.3-0.9 mg/kg, 0.3-0.8 mg/kg, 0.3-0.7 mg/kg, 0.3-0.6 mg/kg, 0.3-0.5 mg/kg, 0.3-0.4 mg/kg, 0.4-1 mg/kg, 0.4-0.9 mg/kg, 0.4-0.8 mg/kg, 0.4-0.7 mg/kg, 0.4-0.6 mg/kg, 0.4-0.5 mg/kg, 0.5-1 mg/kg, 0.5-0.9 mg/kg, 0.5-0.8 mg/kg, 0.5-0.7 mg/kg, or 0.5-0.6 mg/kg. In some embodiments, a MEK inhibitor is administered at a dose of 0.3 mg/kg, 0.4 mg/kg, 0.5 mg/kg, 0.6 mg/kg, 0.7 mg/kg, 0.8 mg/kg, 0.9 mg/kg, or 1 mg/kg.

In some embodiments, a MEK inhibitor (e.g., trametinib) is administered at a dose of 0.5 mg to 5 mg. For example, a MEK inhibitor may be administered at a dose of 0.5-4.5 mg, 0.5-4 mg, 0.5-3.5 mg, 0.5-3 mg, 0.5-2.5 mg, 0.5-2 mg, 0.5-1.5 mg, 0.5-1 mg, 1-5 mg, 1-4.5 mg, 1-4 mg, 1-3.5 mg, 1-3 mg, 1-2.5 mg, 1-2 mg, 1-1.5 mg, 1.5-5 mg, 1.5-4.5 mg, 1.5-4 mg, 1.5-3.5 mg, 1.5-3 mg, 1.5-2.5 mg, 1.5-2 mg, 2-5 mg, 2-4.5 mg, 2-4 mg, 2-3.5 mg, 2-3 mg, 2-2.5 mg, 3-5 mg, 3-4.5 mg, 3-4 mg, 3-3.5 mg, 4-5 mg, 4-4.5 mg, or 4.5-5 mg. In some embodiments, a MEK inhibitor is administered at a dose of 0.5 mg, 1 mg, 1.5 mg, 2 mg, 2.5 mg, 3 mg, 3.5 mg, 4 ng, 4.5 mg, or 5 mg.

In some embodiments, a dose of a MEK inhibitor (e.g., trametinib) is administered as an oral tablet. Other routes of administration, as described below, may be used.

In some embodiments, a dose of a MEK inhibitor (e.g., trametinib) is administered once a day, twice a day, or three times a day, for example, over the course of 10 days, 20 days, 30 days, 60 days, 90 days, 120 days, 150 days, or longer.

In some embodiments, a 2 mg dose of a MEK inhibitor (e.g., trametinib) is administered as an oral tablet.

### SRC Family Inhibitors

The SRC kinase proteins are a family of non-receptor tyrosine kinase proteins which regulate signal transduction pathways involved in cell division, motility, adhesion, and survival. The SRC kinases, including Src, Yes, Fyn, Fgr, Lck, Hck, Blk, Lyn, and Frk, are activated by EGFR, HER2, platelet-derived growth factor receptor (PDGFR), insulin growth factor receptor (IGF-IR), cadherins, and integrins. These activated SRC kinases phosphorylate downstream targets, including protein kinase C (PKC). MAPKs. STAT3, and Akt kinase, thus promoting cell proliferation, survival, and inhibiting apoptosis. A SRC family inhibitor is an agent which inhibits the activation or activity of a SRC family kinase protein. Non-limiting examples of SRC family inhibitors include: KX2-391, bosutinib, saracatinib, PP1, PP2, quercetin, and dasatinib.

In some embodiments, the SRC family inhibitor comprises saracatinib (AZD-0530). Saracatinib is a selective inhibitor of the SRC family of kinase proteins which has been examined for treatment of cancers and Alzheimer's disease. In some embodiments, saracatinib is administered orally as a tablet.

In some embodiments, a SRC family inhibitor (e.g., saracatinib) is administered at a dose of 100 to 1000 mg. For example, a SRC family inhibitor may be administered at a dose of 100-900 mg, 100-800 mg, 100-700 mg, 100-600 mg, 100-500 mg, 100-400 mg, 100-300 mg, 100-200 mg, 200-1000 mg, 200-900 mg, 200-800 mg, 200-700 mg, 200-600 mg, 200-500 mg, 200-400 mg, 200-300 mg, 300-1000 mg, 300-900 mg, 300-800 mg, 300-700 mg, 300-600 mg, 300-500 mg, 300-400 mg, 400-1000 mg, 400-900 mg, 400-800 mg, 400-700 mg, 400-600 mg, 400-500 mg, 500-1000 mg, 500-900 mg, 500-800 mg, 500-700 mg, 500-600 mg, 600-1000 mg, 600-900 mg, 600-800 mg, 600-700 mg, 700-1000 mg, 700-900 mg 700-800 mg, 800-1000 mg, 800-900 mg, 800-700 mg, or 900-1000 mg. In some embodiments, a SRC family inhibitor is administered at a dose of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

mTOR Inhibitors

The mammalian target of rapamycin (mTOR) is a member of the broader PI3K protein kinase family. It integrates the input from numerous upstream pathways, including insulin, growth factors, and amino acids, and regulates critical pathways including cell growth, proliferation, motility, survival, protein synthesis, autophagy, and transcription. mTOR is the catalytic subunit of two distinct protein complexes: mTOR complex 1 (mTORc1) and mTOR complex 2 (mTORc2) complexes. An mTOR inhibitor is an agent which blocks the activity of mTOR or the formation of mTOR complexes. Non-limiting examples of mTOR inhibitors include: rapamycin, sirolimus, temsircimus, everolimus, ridaforolimus, NVPBE235, dactolisib, BGT226, PKI-587, XL765, INK128, sapanisertib. GSK2126458, AZD8055, and AZD2014.

In some embodiments, the mTOR inhibitor comprises rapamycin. In some embodiments, the mTOR inhibitor comprises an analog of rapamycin (rapalog), such as everolimus, sirolimus, temsirolimus, or ridaforolimus. Rapamycin and analogs of rapamycin are approved for treatment of cancers, including advanced renal cell carcinoma (everulimus and temsirolimus), metastatic breast cancer (dactolisib), advanced solid tumors and lymphoma (GSK2126458), glioblastoma multiforme, non-small cell lung cancer, and metastatic breast cancer (XL765), advanced solid tumors and glioma (AZD8055), and advanced solid tumors and multiple myeloma (INK128). In some embodiments, rapamycin and analogs of rapamycin are administered orally (e.g., tablet form).

In some embodiments, an mTOR inhibitor (e.g., rapamycin) is administered at a dose of 5 mg to 20 mg. For example, an mTOR inhibitor may be administered at a dose of 5-15 mg, 5-10 mg, 10-20 mg, 10-15 mg or 15-20 mg. In some embodiments, an mTOR inhibitor is administered at a dose of 5 mg, 10 mg, 15 mg, or 20 mg.

### Combination Therapies

Lapatinib may be administered in combination with one or more of a PI3K pathway inhibitor, a MAPK pathway inhibitor, a SRC family inhibitor, and/or an mTOR inhibitor. In some embodiments, lapatinib and one or more of a PI3K pathway inhibitor, a MAPK pathway inhibitor, a SRC family inhibitor, and/or an mTOR inhibitor are administered simultaneously. In some embodiments, lapatinib and one or more of a PI3K pathway inhibitor, a MAPK pathway inhibitor, a SRC family inhibitor, and/or an mTOR inhibitor are administered sequentially.

The ratio of lapatinib to one or more of a PI3K pathway inhibitor, a MAPK pathway inhibitor, a SRC family inhibitor, and/or an mTOR inhibitor may vary.

In some embodiments, the ratio of lapatinib to PI3K pathway inhibitor (e.g., PI3K inhibitor such as copanlisib) is 1:1 to 1:5. For example, the ratio of lapatinib to PI3K pathway inhibitor may be 1:1, 1:2, 1:3, 1:4, or 1:5. In sore embodiments, the ratio of PI3K pathway inhibitor (e.g., PI3K inhibitor such as copanlisib) to lapatinib is 1:1 to 1:5. For example, the ratio of PI3K pathway inhibitor to lapatinib may be 1:1, 1:2, 1:3, 1:4, or 1:5.

In some embodiments, the ratio of lapatinib to MAPK pathway inhibitor (e.g., MEK inhibitor such as trametinib) is 1:1 to 1:5. For example, the ratio of lapatinib to MAPK pathway inhibitor may be 1:1, 1:2, 1:3, 1:4, or 1:5. In some embodiments, the ratio of MAPK pathway inhibitor (e.g., MEK inhibitor such as trametinib) to lapatinib is 1:1 to 1:5. For example, the ratio of MAPK pathway inhibitor to lapatinib may be 1.1, 1:2, 1:3, 1:4, or 1:5.

In some embodiments, the ratio of lapatinib to Src family inhibitor (e.g., saracatinib) is 1:1 to 1:5. For example, the ratio of lapatinib to Src family inhibitor may be 1:1, 1:2, 1:3, 1:4, or 1:5. In some embodiments, the ratio of Src family inhibitor (e.g., saracatinib) to lapatinib is 1:1 to 1:5. For example, the ratio of Src family inhibitor to lapatinib may be 1:1, 1:2, 1:3, 1:4, or 1:5.

In some embodiments, the ratio of lapatinib to mTOR inhibitor (e.g., rapamycin) is 1:1 to 1:5. For example, the ratio of lapatinib to mTOR inhibitor may be 1:1, 1:2, 1:3, 1:4, or 1:5. In some embodiments, the ratio of mTOR inhibitor (e.g., rapamycin) to lapatinib is 1:1 to 1:5. For example, the ratio of mTOR inhibitor to lapatinib may be 1:1, 1:2, 1:3, 1:4, or 1:5.

In some embodiments, the ratio of lapatinib to copanlisib to trametinib is 1:1:1, 1:2:1, 1:1:2, 1:2:2, 2:1:1, 2:2:1, or 2:1:2.

The dose, dosage, and mutes of administration for each agent (e.g., lapatinib. PI3K pathway inhibitors, MAPK pathway inhibitors, Src family inhibitors, and/or mTOR inhibitors) are described above.

In some embodiments, lapatinib, a PI3K pathway inhibitor (e.g., copanlisib), and a MEK inhibitor (e.g., trametinib) are administered in a therapeutically effective amount to reduce tumor volume in a subject by at least 70%, relative to a control or relative to baseline. For example, lapatinib, a PI3K pathway inhibitor (e.g., copanlisib), and a MEK inhibitor (e.g., trametinib) may be administered in a therapeutically effective amount to reduce tumor volume in a subject by at least 75%, at least 80%, at least 85%, at least 90%, or at least 95% relative to a control or relative to baseline. A control may be an untreated subject or a subject treated with only lapatinib. Baseline, as is known in the art, is the volume of a tumor prior to administration of the particular therapy (e.g., within 1 to 3 months).

### Gastric Cancer Cells

In some embodiments of the present disclosure, gastric cancer cells do not express or express a reduced level of a C-terminal Src kinase (CSK) gene compared to non-cancerous cells. The CSK gene (Gene ID: 1445) encodes the C-terminal Src kinase enzyme. The C-terminal Src kinase enzymes plays an important role in regulating cell growth, differentiation. migration, and the immune response by suppressing the activity of the Src-family kinases, including Src, Hck, Fyn, Lck, Lyn, and Yes1, at tyrosine residues located in the C-terminal end.

In some embodiments, gastric cancer cells do not express or express a reduced level of a phosphatase and tensin homolog (PTEN) gene (Gene ID: 5728) compared to non-cancerous cells. PTEN is a tumor suppressor gene because the encoded PTEN protein is a phosphatase which is involved in regulation of the cell cycle, preventing cells from growing or dividing too rapidly. Specifically, the PTEN protein functions as a tumor suppressor by negatively regulating the PI3K/Akt signaling pathway which promotes cell growth and proliferation.

In some embodiments of the present disclosure, gastric cancer cells do not express or express a reduced level of a CSK gene and do not express or express a reduced level of a PTEN gene compared to non-gastric cancer cells.

In some embodiments, the gastric cancer cells do not express or express a reduced level of CSK, PTEN, BAX, KCTD5, KEAP1, NF1, and TADA) compared to non-cancerous cells.

Some aspects of the present disclosure provide methods that include contacting gastric cancer cells with lapatinib and with a SRC family inhibitor, an mTOR inhibitor, a PI3K pathway inhibitor, a MAPK pathway inhibitor, or a combination thereof.

Contacting refers to exposing cells to an agent such as lapatinib, a SRC family inhibitor, an mTOR inhibitor, a PI3K pathway inhibitor, a MAPK pathway inhibitor, or a combination thereof. For example, an agent may be added to or combined with a composition comprising gastric cancer cells or administered to a subject having gastric cancer.

Gastric cancer cells of the present disclosure may be either in vivo or ex vivo (e.g., in vitro). Gastric cancer cell lines may be isolated from primary and/or secondary (e.g., metastatic) tumor sites. Non-limiting examples of gastric cancer cell lines include: OE19, NCI-N87 (N87), KATOIII, SNU-16, SNU-5, AGS, SNU-1, and Hs-746 T. Gastric cancer cells of the present disclosure, in some embodiments, are within a subject having (e.g., diagnosed with) gastric cancer. In some embodiments, a subject is a mammal, optionally a human.

### Kits

Some aspects of the present disclosure provide a kit comprising lapatinib and a PI3K pathway inhibitor, a MAPK pathway inhibitor, or a PI3K pathway inhibitor and a MAPK pathway inhibitor.

In some embodiments, the PI3K inhibitor pathway comprises a PI3K inhibitor. In some embodiments, the PI3K inhibitor comprises copanlisib.

In some embodiments, the MAPK pathway inhibitor comprises a MEK inhibitor. In some embodiments, the MEK inhibitor comprises trametinib.

In some embodiments, the kit comprises lapatinib, copanlisib, and trametinib.

Other components of a kit as provided herein may include deliver devices, such as syringes and needles, carriers, and/or excipients.

### Additional Methods

Some aspects of the present disclosure provide methods comprising delivering to in vitro control cells and to human gastric cancer cells harboring HER2 amplification a pooled genome-scale CRISPR-Cas9 knockout library, treating the control cells and gastric cancer cells with lapatinib, extracting the DNA from the lapatinib-treated control and lapatinib-treated gastric cancer cells, sequencing the DNA extracted from the lapatinib-treated cells, and identifying from the sequenced DNA candidate loss-of-function genes that may contribute to lapatinib resistance.

Delivering refers to the targeted entry of packaged nucleic acids into cells. In some embodiments, delivering is by a lentiviral delivery system. A lentiviral delivery system, in some embodiments, comprises a lentiviral transfer plasmid encoding the transgene of interest to be integrated into the host cell genome, a packaging plasmid, and an envelope plasmid. The lentiviral transfer plasmid, in some embodiments, also comprises long terminal repeat sequences, which facilitate integration of the transfer plasmid into the host cell genome. Once integrated into the host cell genome, the transgene from the lentiviral transfer plasmid is expressed, along with the packaging and envelope plasmids. The transgene of interest is then packaged into lentiviral particles, which are used to deliver the transgene of interest into a cell. Lentiviral delivery systems integrate a transgene of interest into both dividing and non-dividing cells and are commonly utilized in vitro.

In some embodiments, methods of the present disclosure use a library of CRISPR-Cas9 genome-wide guide RNAs (gRNAs). The clustered regularly interspaced short palindromic repeats (CRISPR)-Cas system is a naturally occurring defense mechanism in prokaryotes which has been repurposed as a RNA-guided DNA-targeting platform useful in gene editing. It relies on the DNA nuclease Cas9, and two noncoding RNAs—crisprRNA (crRNA) and a trans-activating RNA (tracrRNA)—to target the cleavage of DNA.

In some embodiments of the present disclosure, a CRISPR-Cas9 library comprising single guide RNAs (gRNAs) which target thousands of genes in the human genome is delivered to either human control cells or human HER2-amplified gastric cancer cells. These gRNAs, in combination with the Cas9 nuclease, facilitate the knock-out of genes throughout the human genome. In some embodiments, the delivering is by a lentiviral delivery system.

In some embodiments, human control cells or human HER2-amplified gastric cancer cells comprising the CRISPR-Cas9 genome-wide library are treated with the HER2 inhibitor lapatinib. As described above, lapatinib is a HER2 inhibitor approved for the treatment of HER2-amplified metastatic breast cancer. Lapatinib has also been investigated as a therapy for HER2-amplified gastric cancer cells, but it fails to prolong the survival of subjects. Therefore, it is likely that at least one gene promotes resistance of HER2-amplified gastric cancer cells to lapatinib compared to HER2-amplified breast cancer cells.

In some embodiments, following lapatinib treatment, the DNA is extracted from control cells and HER2-amplified gastric cancer cells. DNA extraction is the process of purifying the DNA from a cell. Numerous methods for extracting DNA exist, which comprise the common steps of lysing the cells, concentrating the DNA, and purifying the DNA.

In some embodiments, the extracted DNA is sequenced. Non-limiting examples of sequencing which may be utilized include: deep sequencing, massively parallel signature sequencing (MPSS), polony sequencing, 454 pyrosequencing, Illumina (Solexa) sequencing, combinatorial probe anchor synthesis (cPAS), SOLiD sequence, Ion Torrent semiconductor sequencing. DNA nanoball sequencing, Heliscope single molecule sequencing, single molecule real time (SMRT) sequencing, and Nanopore DNA sequencing.

In some embodiments, following sequencing, the expression of genes in lapatinib-treated control cells are compared to the expression of genes in lapatinib-treated HER2-amplified gastric cancer cells. Methods for comparing the gene expression of control and HER2-amplified gastric cancer cells include the use of different algorithms, including Model-based Analysis of Genome-wide CRISPR-Cas9 Knockout robust rank aggregation (MAGeCK RRA), MAGeCK MLE, edgeR, and dynamic programming (DP).

Comparing the expression of genes in the lapatinib-treated control cells and HER2-amplified gastric cells may lead to candidate loss-of-function genes whose expression is absent or downregulated in HER2-amplified gastric cancer cells compared to control cells. In some embodiments, candidate loss-of-function genes can be validated by delivering to control cells and HER2-amplified gastric cancer cells a gRNA which targets the candidate loss-of-function gene, treating the cells with lapatinib, and assessing cell viability to evaluate if the loss-of-function gene confers lapatinib resistance.

Cell viability can be monitored utilizing either cell survival or apoptosis assays. Cell viability assays, which include clonogenic assays, propodium iodine assays, TUNEL assays, and Trypan Blue assays, determine the ability of cells to maintain or recover viability following treatment. Apoptosis assays, which include caspase activation, cleavage of Bcl-2 proteins, caspase substrate cleavage, mitochondrial transmembrane potential, and cytochrome C release, determine the presence or degree of cell death following treatment.

In some embodiments of the present disclosure, the viability of lapatinib treated-control cells or HER2 amplified gastric cancer cells is assessed by measuring caspase activation. In some embodiments, the caspase is caspase 3. In some embodiments, the caspase is caspase 7. In some embodiments the caspase is caspase 3 and caspase 7.

### EXAMPLES

### Example 1. CRISPR Library Screening Identified Candidate Genes Whose Loss of Function Confer Lapatinib Resistance in HER2-Amplified GC Cells

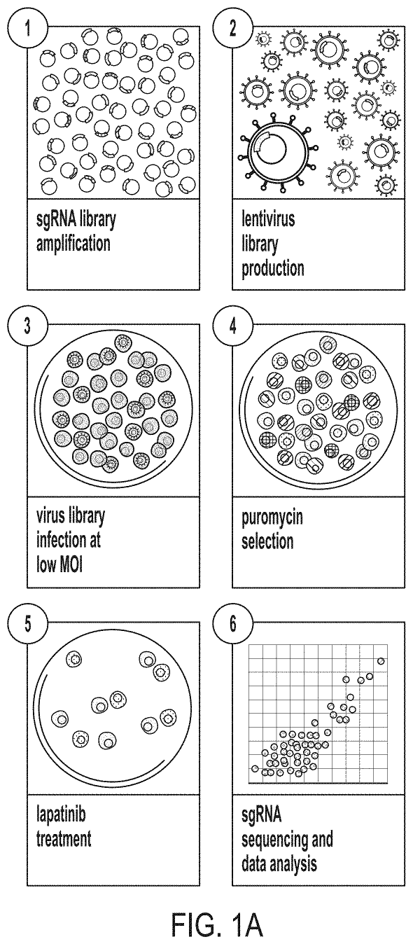

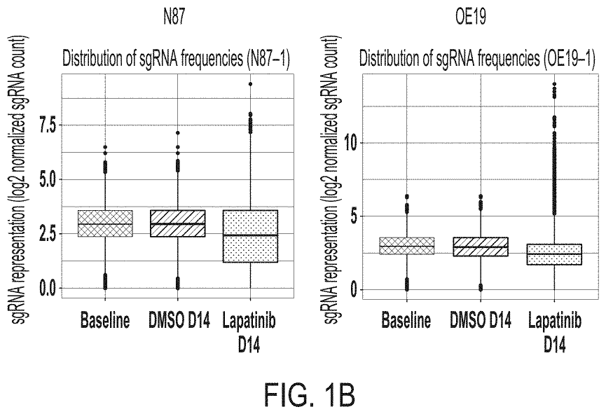

To identify genes whose loss of function confer drug resistance to Lapatinib, we performed a genome-wide CRISPR/Cas9 gene knockout screening in two human GC cell lines harboring HER2 amplification, N87 and OE19, respectively. As shown in the schematic diagram of CRISPR screening (FIG. 1A), pooled GeCKO V2 library were amplified for lentivirus production, and then two biological replicates of N87 and OE19 cells were transduced with the lentivirus containing GeCKO V2 library at multiplicity of infection (MOI) of 0.3. Puromycin selection was performed before Lapatinib treatment on each cell line. After 14 days of Lapatinib treatment, the drug treated and vehicle treated cells were harvested. The genomic DNA was extracted for PCR amplification and subsequent deep sequencing of the regions containing the gRNAs.

The deep sequencing data showed that the gRNA distribution from the Lapatinib treated cells was significantly different from the vehicle treated cells in both N87 and OE19 cell lines (Wilconxon rank-sum test, p-value <2.2e-16) (FIG. 1B). The replicates of Lapatinib treated cells are clustered separately from other conditions and all replicates within samples are highly correlated (Pearson correlation coefficient >0.9) (data not shown), indicating the consistency of our screening system. In addition, we found enrichments of multiple gRNAs in the Lapatinib treated cells by analyzing the read count changes for each gRNA in Lapatinib treatment samples relative to the control samples. After 14 days Lapatinib treatment, 694 and 3 gRNAs were greater than 20-fold and 100-fold enrichment in N87 group, and 357 and 34 gRNAs were greater than 20-fold and 100-fold enrichment in OE19 group, respectively. A panel of candidate genes was detected from this screening using three different algorithms. MAGeCK RRA. MAGeCK MLE and edgeR, and the top enriched genes including CSK, BAX, KEAP1, PRR24, TADA1, KCTD5, PTEN and NF1 etc. are highlighted in FIG. 1C. Our data indicates that loss of these particular genes may contribute to Lapatinib resistance.

### Example 2. In Vitro Validation Study Confirmed Low of Function of CSK, PTEN and Other Candidate Genes Confer the Resistance to Lapatinib

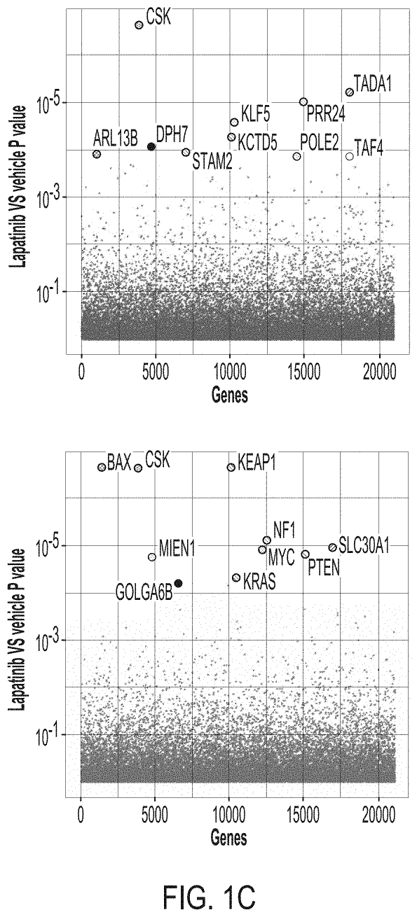

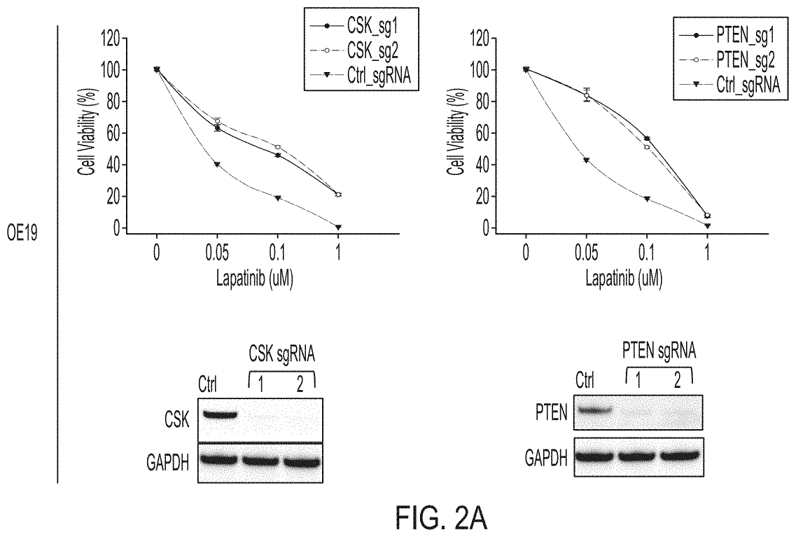

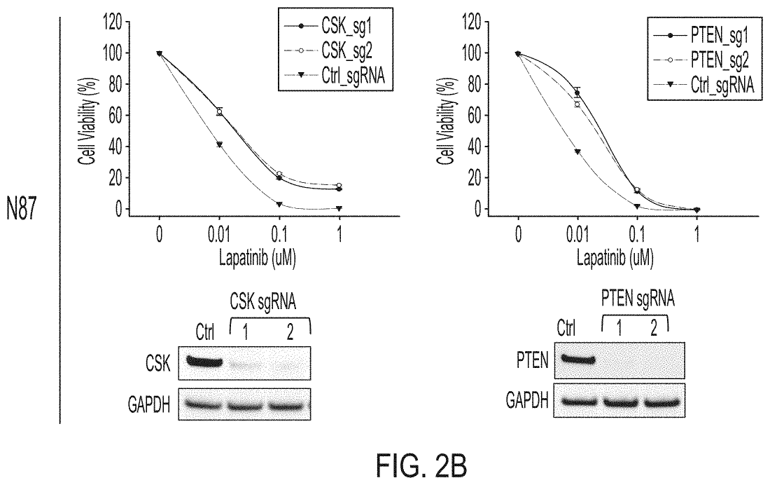

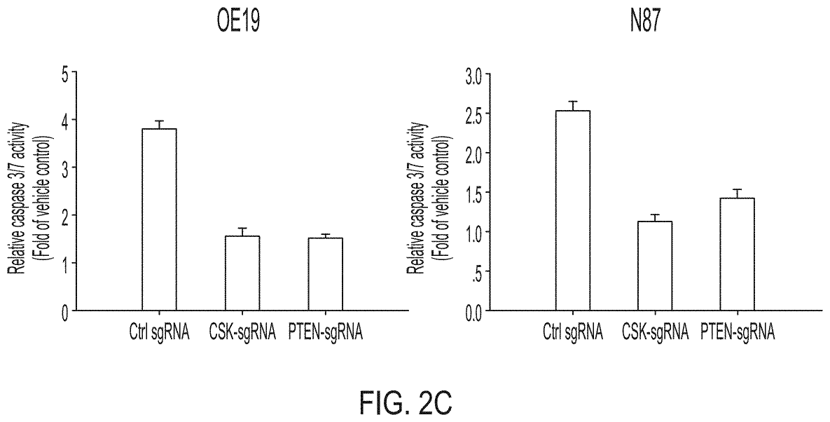

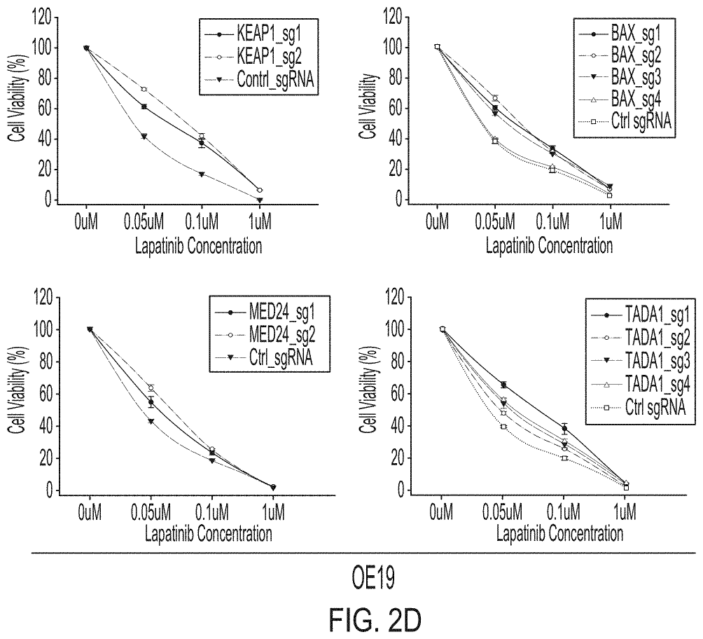

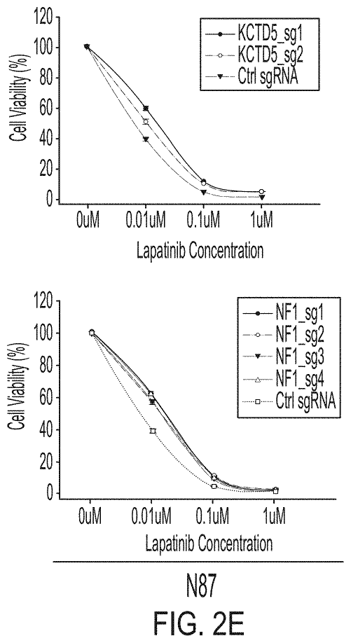

After identifying candidate genes from the screening described above, we then performed validation experiments on selected genes to check whether loss of function of these genes confer Lapatinib resistance in the GC cells. The genes selected for validation include: I) Genes were identified as the top 20 candidates by at least 2 out of 3 algorithms (MAGeCK RRA, MAGeCK MLE and edgeR; 2) genes were identified as the top 20 candidates in both N87 and OE19 cells. Two gRNAs for each gene were picked for validation. N87 and OE19 cells were infected with lentivirus carrying gene targeting gRNAs and treated with various doses of Lapatinib. Cell viability was determined after 6 days to evaluate the drug resistance. While a significant resistance to Lapatinib was validated in OE19 cells with loss of function of BAX, KEAP1, NF1 and TADA1, as well as in N87 cells with loss of function of NF1 and KCTD5, respectively (FIGS. 2D and 2E), our data showed that loss of CSK or PTEN in N87 and OE19 cells (confirmed by Western blotting) conferred the most significant resistance to Lapatinib treatment (FIGS. 2A and 2B).

Both CSK and PTEN are tumor suppressor genes. CSK is a ion-receptor protein tyrosine kinase that serves as an indispensable negative regulator of the SRC Family tyrosine Kinases (SFKs). An up-regulation of SRC signaling has been linked to cancer progression by promoting other signals¹⁷. PTEN is a protein tyrosine phosphatase that negatively regulates PI3K/AKT pathway to repress tumor cell growth and survival. Since loss of CSK or PTEN exhibited the most significant resistance to Lapatinib in both N87 and OE19 cells, we subsequently focused on the characterization of CSK and PTEN null cells in this study.

We examined apoptosis induced by Lapatinib treatment in CSK or PTEN null cells as well as the control cells by measuring caspase-3/7 activation (Caspase-Glo assay). Consistent with the cell viability result, the OE19 cells transduced with non-targeting gRNA showed 3.79±0.16 fold of caspase-3/7 activity compared with vehicle treated cells, while CSK or PTEN null OE19 cells presented 1.55±0.16 and 1.50±0.10 fold of caspase-3/7 activity. respectively (FIG. 2C). Similar result was obtained from the experiment with CSK or PTEN null N87 cells. The results indicate that loss of function of CSK or PTEN significantly inhibited the Lapatinib-induced apoptosis.

### Example 3. Up-Regulation of PI3K and MAPK Pathways in CSK or PTEN Null GC Cells with Lapatinib Resistance

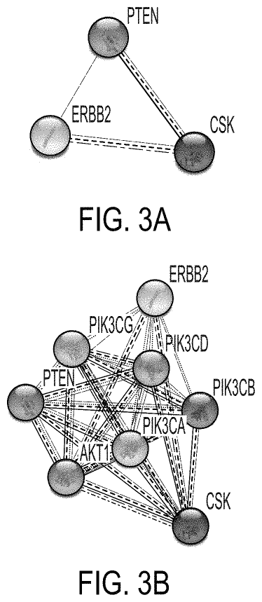

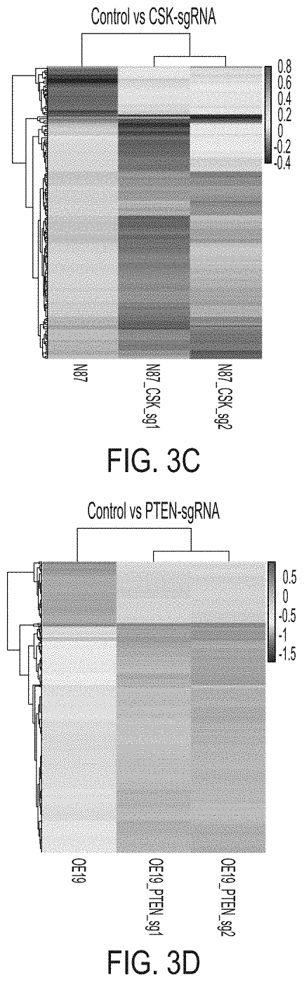

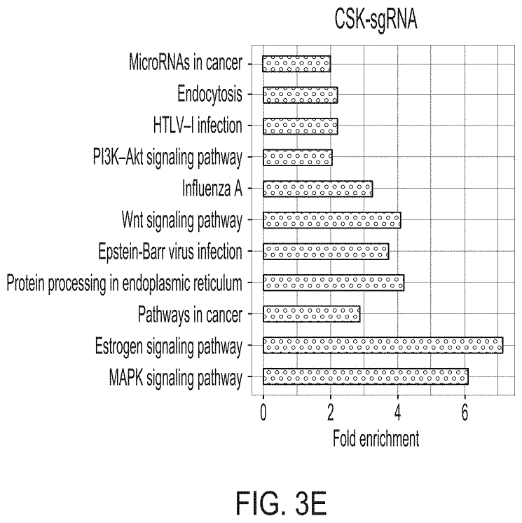

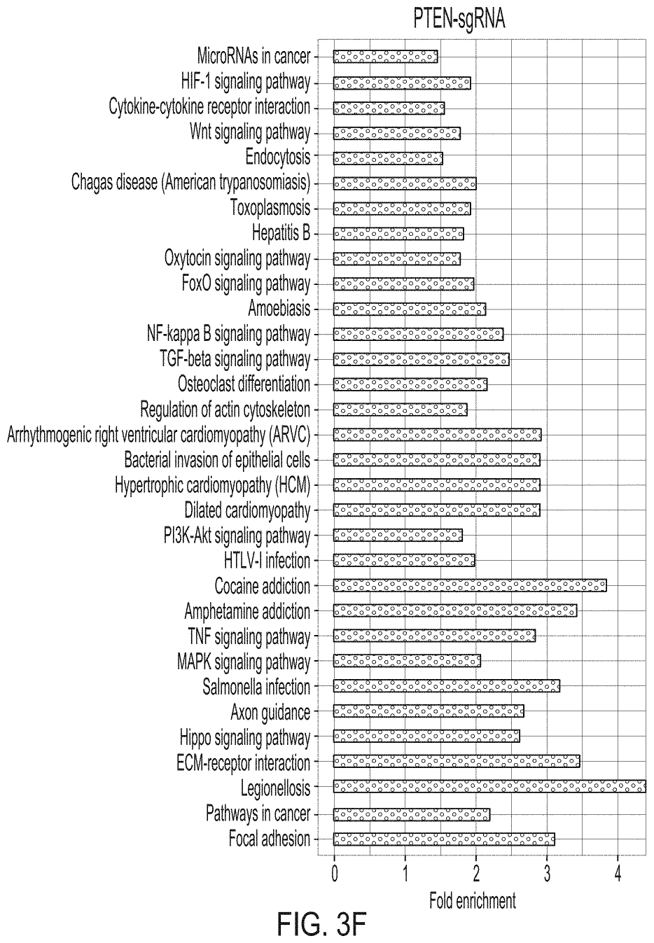

The similar resistance phenotype of CSK and PTEN knockouts suggests that these two genes might be functionally linked. To further understand whether there is functional interaction between these two genes, we analyzed the protein interaction networks by Search Tool for the Retrieval of Interacting Genes (STRING)¹⁸. The networks formed by interacting proteins are helpful in understanding potential molecular mechanisms and predicting potential partners of Lapatinib resistance in HER2 amplified GC cells. Here, the STRING analysis suggests that CSK, PTEN and ERBB2 (HER2) are functionally linked (FIG. 3A) and PI3K/AKT pathway components PIK3CA, AKT1, PIK3CG, PIK3CB, PIK3CD are predicted as the directly linked functional partners (FIG. 3B), indicating that CSK and PTEN may be involved in HER2 signaling and PI3K/AKT pathway in GC cells. To better understand the molecular mechanism of Lapatinib resistance in CSK or PTEN null GC cells, we performed the RNA-Seq on CSK or PTEN null cells and the corresponding parental cells to examine the difference of their transcriptome profiles. The differentially expressed genes (DEGs) identified by DESeq2 are shown in the heat map (FIGS. 3C and 3D). A total of 139 genes between CSK null cells and parental (N87) cells and 997 genes between PTEN null cells and parental (OE19) cells were detected as significant DEGs (Fold change >1.5, FDR<0.1). The data also indicates that loss of PTEN mutation have much higher impact on gene expression profile than loss of CSK in GC cells. To provide insight into the cellular pathways associated with these genes, Kyoto Encyclopedia of Genes and Genomes (KEGG) pathway analysis of the DEGs was performed. Among the pathways present in FIGS. 3E and 3F, we found that MAPK, PI3K, and Wnt pathways were commonly enriched in both CSK and PTEN null GC cells (>1.5 fold enrichment), suggesting that these pathways may play important roles in Lapatinib resistance.

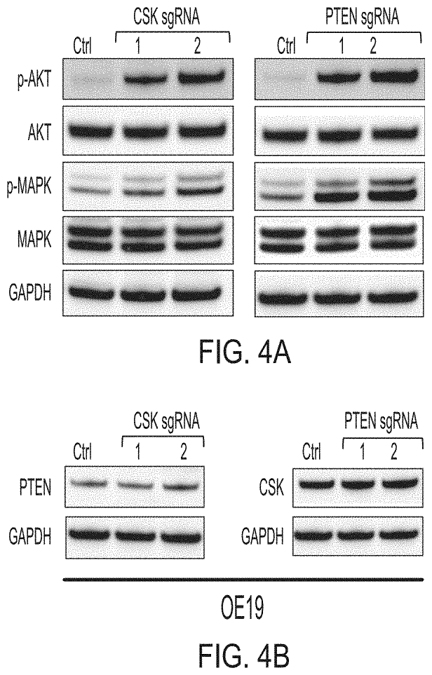

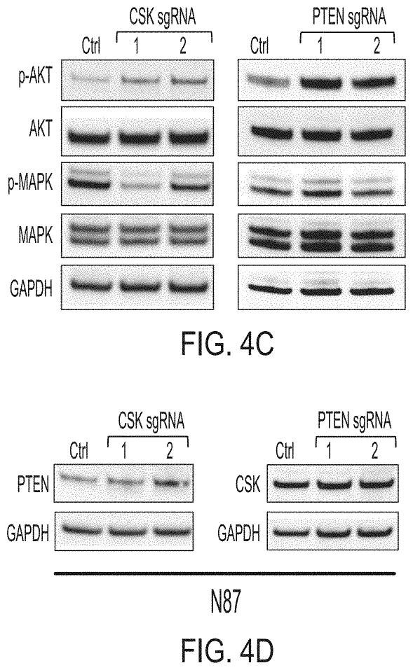

Considering that PI3K and MAPK are the major pathways downstream of HER2 receptor, we hypothesized that loss of CSK or PTEN function would up-regulate the PI3K/AKT and/or MAPK signaling pathways, thereby causing cells to be resistant to Lapatinib. To further test our hypothesis, we examined the phosphorylation levels of AKT and MAPK in CSK or PTEN null GC cells by Western blotting, respectively. Consistent with the results of protein interaction and pathway analysis, we found that the phosphorylation level of AKT and MAPK dramatically increased in CSK or FEN null cells compared with the control OE19 cells (FIG. 4A). A similar pattern of increased phosphorylation of AKT were observed in the CSK or PTEN knockout N87 cells, but phosphorylation level of MAPK seems decreased or no significant change compared with the control cells (FIG. 4C). In addition, we measured the PTEN expression in CSK knockout OE19 and N87 cells and CSK expression in PTEN knockout OE19 and N87 cells by Western blotting. No significant change of CSK protein expression was observed in PTEN null cells and no significant change of PTEN protein expression was shown in CSK null cells (FIGS. 4B and 4D), suggesting that CSK and PTEN do not regulate the protein level of each other.

### Example 4. Pharmacological Inhibition of PI3K and MAPK Pathways Synergistically Overcome the Resistance to Lapatinib in CSK or PTEN Null GC Cells

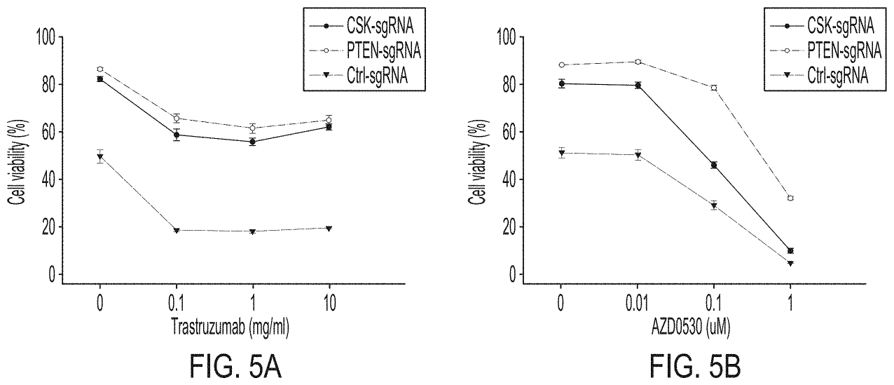

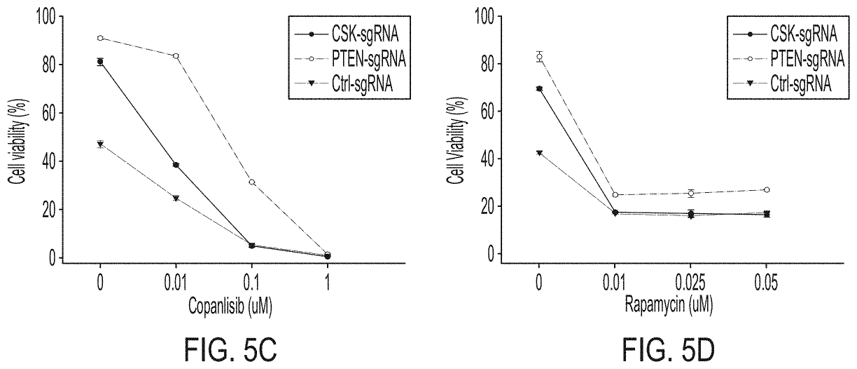

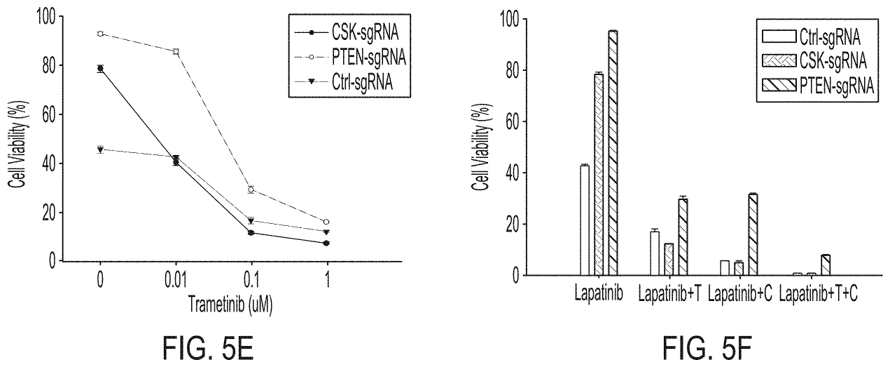

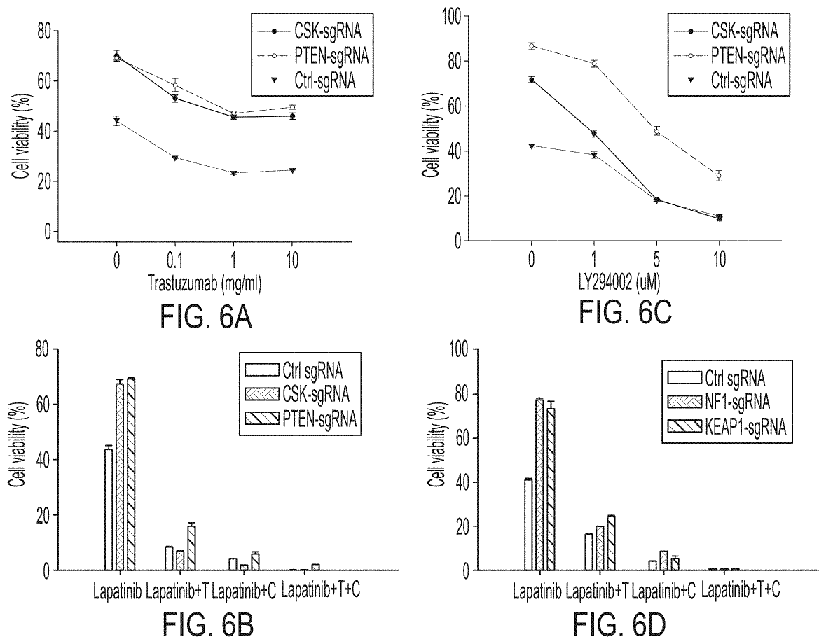

Trastuzumab is a potent anti-HER2 agent and is usually applied with or without Lapatinib in HER2 amplified breast cancer patients clinically. And Trastuzumab based treatment has been approved by FDA as a target treatment for HER2-positive advanced GC¹⁹. Here, we tested whether loss of CSK or PTEN in GC cells confer resistance to the combination of Trastuzumab and Lapatinib. We observed that CSK or PTEN null OE19 cells were significantly resistant to combination of Lapatinib (0.05 μM) and Trastuzumab (0.1-10 mg/ml) compared with the control cells, although the combination of Trastuzumab and Lapatinib showed some inhibitory effect on CSK or PTEN null OE19 cells (FIG. 5A). And similar result was obtained in CSK or PTEN null N87 cells (FIG. 6A), indicating that loss of function of CSK or PTEN confers resistance to both Lapatinib and Trastuzumab in HER2 amplified GC cells.

To further test our hypothesis that re-activation of the signaling downstream of HER2 is the underlying mechanism for the Lapatinib resistance, we employed a pharmacological approach to modulate PI3K/AKT/mTOR, MAPK and SRC signaling pathways. Since CSK negatively regulates SRC signaling, we first treated CSK and PTEN null OE19 cells with the SRC family kinase inhibitor AZD0530 (Saracatinib). As shown in FIG. 5B. AZD0530 in combination with 0.05 μM Lapatinib decreased the cell viability of CSK or PTEN null OE19 cells in a dose-dependent manner (0.01-1 μM), indicating that re-activation of the SRC signaling pathway in both CSK and PTEN null OE19 cells may confer the resistance to Lapatinib. However, 42.49±2.54% PTEN null cells still survived while only 9.15±0.88% of CSK null cells survived the AZD0530 treatment at the highest concentration (1 μM), suggesting that the SRC signaling might be the major re-activated pathway in the CSK knockout OE19 cells, but not in the PTEN knockout OE19 cells. In addition, we treated the CSK and PTEN null OE19 cells with the PI3K inhibitor Copanlisib (BAY 80-6946), a drug that was approved by FDA for patients with relapsed follicular lymphoma²⁰). Remarkably. Copanlisib (0.01-1 μM) in combination with 0.05 μM Lapatinib inhibited the cell growth of CSK or PTEN null OE19 cells as well as control cells in a dose-dependent manner (FIG. 5C), and similar results were obtained with another PI3K inhibitor LY294002 (FIG. 6C), indicating PI3K pathway plays an important rule in Lapatinib resistance of CSK or PTEN null GC cells. Since mTOR is a key molecule downstream of PI3K pathway that regulates cell growth, proliferation and survival, we tested whether mTOR inhibitor Rapamycin could overcome the Lapatinib resistance in the CSK or PTEN null GC cells. We found that Rapamycin (0.01 μM) in combination with Lapatinib (0.05 μM) significantly inhibited cell growth of CSK or PTEN knockout OE19 cells as well as control cells. However, increased doses of Rapamycin (0.025-0.05 μM) with 0.05 μM Lapatinib couldn't further decrease cell growth (FIG. 5D), suggesting that mTOR may only partially contribute to the Lapatinib resistance in the GC cells. Moreover, we treated the CSK and PTEN null OE19 cells with Lapatinib and the MAPK inhibitor Trametinib (GSK1120212), a drug that was approved by FDA in combination with Dabrafenib for the treatment of patients with BRAF V600E/K-mutant metastatic melanoma²¹. In our pharmacological test, we observed that Trametinib (0.01-1 μM) in combination with 0.05 μM Lapatinib dramatically overcome the resistance to Lapatinib in the CSK or PTEN null OE19 cells in a dose-dependent manner (FIG. 5E), indicating that re-activation of MAPK pathway in CSK or PTEN null OE19 cells may play important role in Lapatinib resistance.

While the combination of Lapatinib with Copanlisib or Trametinib could significantly inhibit the Lapatinib resistance in the CSK or PTEN null OE19 cells, it request high dose of drugs (1 μM Copanlisib or 1 μM Trametinib) to fully overcome the resistance (FIGS. 5C and 5E). Therefore, we tested another treatment strategy using a combination of Lapatinib. Copanlisib and Trametinib with relative low doses. As shown in FIG. 5F, Lapatinib alone could not inhibit the growth of CSK or PTEN null OE19 cells. However. Lapatinib in combination with 0.1 μM Copanlisib or 0.1 μM Trametinib significantly inhibited the cell growth, but not completely. Furthermore, when the cells were treated with a combination of these three agents (i.e., 0.05 μM Lapatinib, 0.1 μM Copanlisib, and 0.1 μM Trametinib), the cell viability dramatically decreased to 0.22±0.08% in CSK null OE19 cells and 7.65±0.31% in the PTEN null OE19 cells, respectively. It almost completely overcame the Lapatinib resistance. Similar result was present in CSK or PTEN null N87 cells (FIG. 6B). This finding indicates that re-activation of PI3K and MAPK are both involved in Lapatinib resistance in HER2 amplified GC with loss of function of CSK or PTEN. Thus, our study provides a feasible therapeutic strategy for the GC patients of HER2 amplification with CSK or/and PTEN loss of function mutation.

### Example 5. In Vivo Test of Treatment Strategy to Overcome Lapatinib Resistance in HER2 Amplified Gastric Cancer with CSK or PTEN Mutation

The goal of this In vivo study is to further validate the efficacy of drug combination of lapatinib, copanlisib (PI3K inhibitor), and trametinb (MEK inhibitor) in HER2 amplified gastric cancer with loss of function mutations of CSK or PEN by using N87-CSK^{-/-} and N87-PTEN^{-/-} mouse xenograft tumor models.

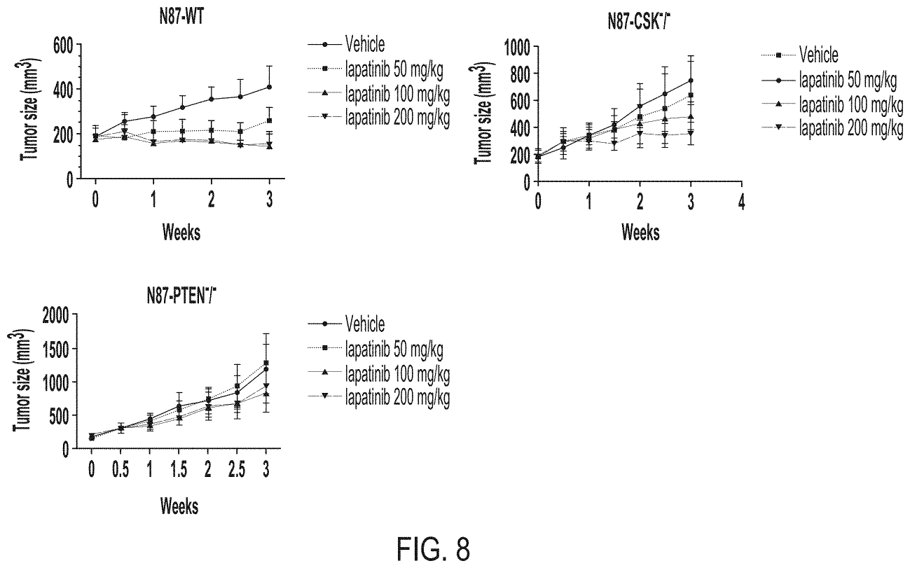

In our first experiment, we did a lapatinib dosing test with N87-WT, N87-CSK^{-/-} and N87-PTEN^{-/-} xenograft tumor models. N87-WT tumors grow relatively slow and are sensitive to lapatinib treatment. N87-CSK^{-/-} and N87-PTEN^{-/-} tumors grow much faster and form big tumor masses after three (3) weeks of treatment. Compared to N87-WT rumors. N87-CSK^{-/-} tumors are relatively insensitive to lapatinib, and N87-PTEN^{-/-} tumors are resistant to lapatinib treatment (FIG. 8).

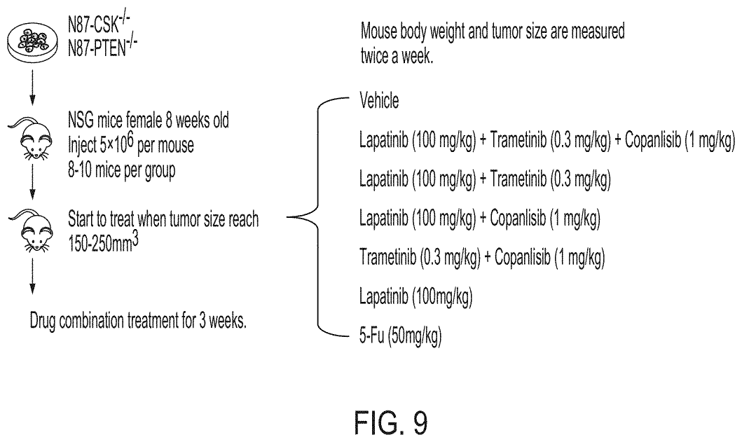

In the second experiment, we compared the efficacy of lapatinib+trametinib+copanlisib with other treatment conditions including gastric cancer standard chemotherapy agent fluorouracil (5-FU) (FIG. 9).

In Vivo Pharmacological Assessment with Xenograft Model

Six-to-seven-week-old female NOD/SCID/IL-2γ-receptor null (NSG) female mice were purchased. The initial body weight of the animals at the time of arrival was between 19 and 22 g. Mice were allowed to acclimatize to local conditions for 1 week before being injected with tumor cells. Tumors were induced by injecting N87-WT. N87-CSK-r or N87-PTEN^{-/-} cells (5×10⁶) subcutaneously into the right flank of mice. The tumors were then measured twice a week using calipers, and the tumor volume in mm³ was calculated according to following formula: (width²×length)/2. Drug treatment was initiated when tumors reached a volume of 150-250 mm³. Mice were randomly divided into seven treatment groups including 8-10 mice in each group: 1) vehicle only, 2) lapatinib only, 3) lapatinib+trametinib, 4) lapatinib+copanlisib, 5) trametinib+copanlisib, 6) lapatinib+trametinib+copanlisib, and 7) 5-FU. Lapatinib was administered via oral gavage at a concentration of 100 mg/kg in 2% DMSO, 30% polyethylene glycol (PEG) 300 (Sigma), 5% Tween 80 (Sigma) in sterile Milli-Q water Monday through Friday. A dose of 50 mg/kg of 5-FU in was given intraperitoneally once weekly. Trametinib was administered by oral gavage at concentration of 0.3 mg/kg in 30% PEG400 and 0.5% DMSO in sterile Milli-Q water Monday through Friday. Copanlisib was administered by intravenous injection at the dose of 1 mg/kg in 20% PEG 400/acidified water (0.1N HC, pH 3.5) three times weekly. After 21 days of treatment. the animals were euthanized and the tumors were collected from each mouse to measure the weight. Results are presented as mean volumes or weights for each group. Error bars represent the SD of the mean. Statistical calculations were performed using Prism 8 (GraphPad). Statistical analysis to compare tumor volumes in xenograft-bearing mice was performed with ANOVA. Differences between two groups of tumor mass were assessed by an unpaired Student's t test. Differences between groups were considered statistically significant if P<0.05.

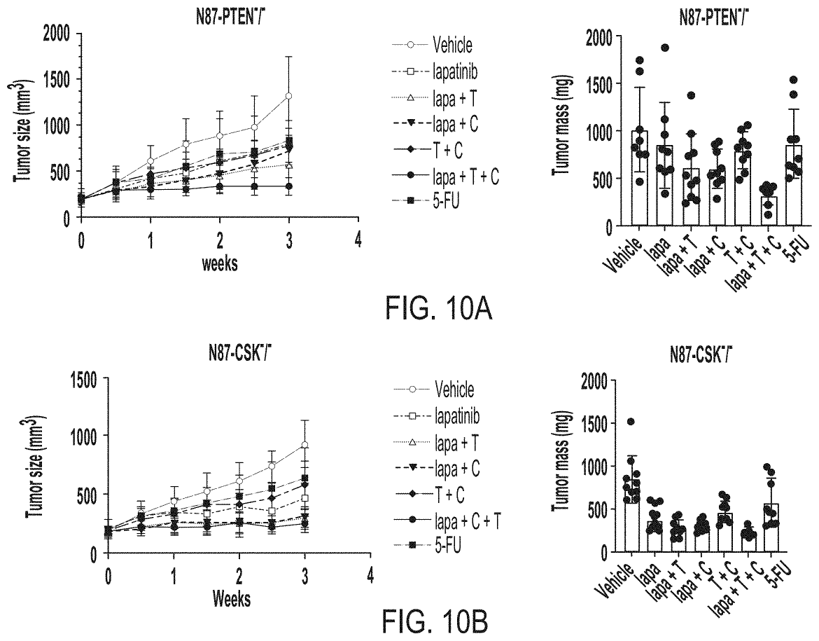

From the in vivo test with N87-PTEN-r xenograft tumor, a significant effect upon tumor growth was observed with the combination of lapatinib, trametinib and copanlisib (2-way ANOVA: ***, P<0.0001) when compared with vehicle, lapatinib alone or 5-FU treatment groups, respectively. Similarly, when the mass of the tumors at endpoint were compared, the three-drug combinations showed significant improvement over lapatinib alone (Unpaired t test: ***, P=0.0008) and 5-FU (Unpaired t test: **, P=0.0013) Error bars. SD (FIG. 10A). Similar result was obtained from the experiment with N87-CSK⁻/⁻ xenograft (FIG. 10B). N87-CSK-r tumors seem less resistant to lapatinib treatment than N87-PTEN⁻/⁻. Consistent with our in vitro study result, the in vivo drug treatment study suggests that lapatinib combined with trametinib and copanlisib can significantly inhibit tumor growth in those lapatinib resistant tumors with loss of function mutations of CSK or PTEN. This drug combination potentially will be effective on lapatinib resistant HER2-amplified gastric cancer with other related genomic alterations in PI3K or MAPK pathways.

Molecular targeted therapy has shown great specificity in eliminating malignant cells with minimal side effects in cancer therapy compared with conventional chemotherapy²². Lapatinib, a dual EGFR and HER2 inhibitor, are clinically effective against HER2 amplified breast cancer by blocking HER2 phosphorylation, resulting in inhibition of downstream PI3K/AKT and MAPK pathways²³. However, it didn't improve survival significantly in clinical trials of HER2 amplified GC, indicating additional oncogenic alterations could contribute to the drug resistance in GC. The previous studies in GC suggest that signaling through other receptor tyrosine kinases (RTKs), such as amplification of MET, IGFR, and HER3 confer anti-HER2 treatment resistance by re-stimulating downstream PI3K and MAPK signal transduction, thus bypassing the inhibitory effect of Lapatinib or Trastuzumab²⁴⁻²⁵. In our CRISPR/Cas9 based genome-wide knockout screening study, we identified and demonstrated that loss of function mutations of CSK or PTEN conferred resistance to Lapatinib in HER2 amplified GC cell lines by restoring downstream PI3K and MAPK pathways of HER2 receptor. Interestingly, previous study in breast cancer suggests that increased SRC kinases activates the PI3K signaling cascade via altering the capacity of the PTEN C2 domain binding to the cellular membranes rather than directly interfering with PTEN enzymatic activity²⁶. Combining with our observation, it indicates CSK, the SRC family kinases negative regulator, functionally linked with PTEN in regulating PI3K signaling in Lapatinib resistance, which explained the similar resistance phenotype of CSK and PTEN null GC cells and stable PTEN protein level in CSK null cells in our study (FIGS. 2A-2C and FIGS. 4A-4D). In addition, our result is supported by previous study in breast cancer that PTEN loss triggered hyperactivation of the MAPK pathway²⁷. Taken together, our data suggest that loss of function mutation of CSK or PTEN may lead to the up-regulation and hyperactivation of PI3K and MAPK pathways, which could be the central mechanism for Lapatinib resistance in these GC cells.

Wa also showed that PI3K inhibitor and MEK inhibitor may increase the sensitivity of the resistant GC cells to Lapatinib. This finding could be potentially important for developing novel anti-HER2 therapy. In particular, HER2 amplified GC patients with CSK or PTEN mutation might therefore be good candidates for combinational therapy with Lapatinib. PI3K inhibitor and MEK inhibitor. To explore the potential clinical application, we checked the status of PTEN or CSK mutations in the HER2 amplified GC cases. For this purpose, we collected the variants data from over 3,000 GC patient samples from the TCGA (The Cancer Genome Atlas) and other cohorts²⁸, and 103 GC patient samples from our previous study²⁹ (Table 1). Over 25% of GC patients showed HER2 amplification in the TCGA samples. Approximately 14% of GC patients harbored HER2 amplification in our smaller dataset. Interestingly, 3-14% of the GC patients with HER2 amplification have either PTEN or CSK mutations in the genome. Of note, the TCGA and other cohort data is only based on somatic mutations and this percentage will be increased if we include genii line mutations. In addition, GC patients with HER2 amplification and gain of function mutations in PIK3CA could also benefit from this treatment strategy since gain of function mutations in PIK3CA have been suggested to be associated with Trastuzumab/Lapatinib resistance by up-regulating PI3K pathway in breast cancer³⁰⁻³¹.

TABLE 1
PTEN and CSK mutation profiles in the HER2 amplified gastric
cancer patients

| | | | | Samples with HER amplifica- | |
|---|---|---|---|---|---|
| | | Samples with HER2 | | tion + PTEN/CSK mutation | |
| | Sample | amplification | | | Percentage in HER2 |
| Data Set | Size | Samples | Percentage | Samples | amplified patients |
| TCGA | 3,089 | 772 | 25% | 21 | 3% |
| somatic | | | | | |
| Park et al | 103 | 14 | 14% | 2 | 14% |
| (PNAS | | | | | |
| 2015) | | | | | |

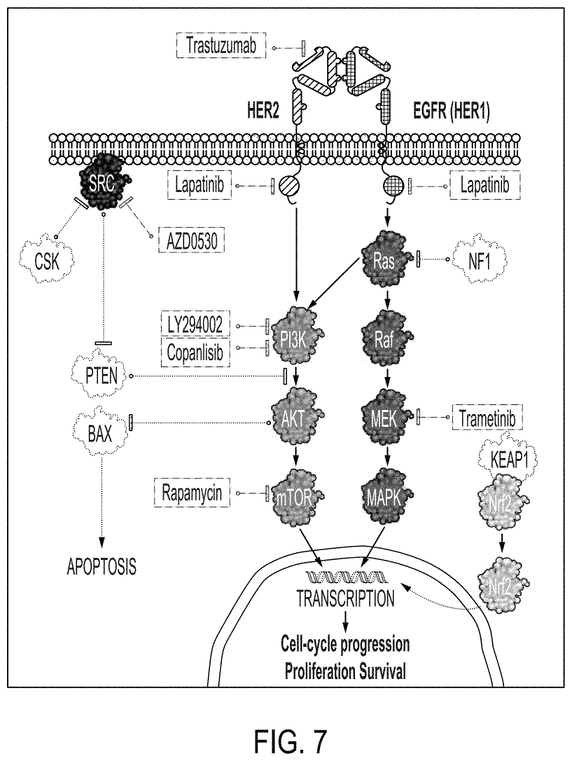

In this study, we also identified and validated other genes that may be involved in Lapatinib resistance, such as NF1 and KEAP1 (FIGS. 2D and 2E) Interestingly, we found that Lapatinib in combination with PI3K inhibitor Copanlisib and MEK inhibitor Trametinib could also overcome the resistance to Lapatinib conferred by NF1 and KEAP1 knockout (FIG. 6D). Although the mechanism is not elucidated in GC, loss of NF1 has been associated with resistance to EGFR TKIs in lung adenocarcinomas and resistance to BRAF inhibitor in melanoma by increasing MAPK and/or PI3K signaling via negatively regulating Ras³²⁻³³. Previous studies suggest that loss of KEAP1 function may lead to the nuclear translocation of Nrf2 and subsequent increased expression of cellular antioxidants and xenobiotic detoxification enzymes, which may be the major resistance mechanism of tumor cells against chemotherapeutic drugs³⁴⁻³⁵. Interestingly, Nrf2 activation caused by loss of KEAP1 could be blocked by PI3K inhibitor³⁶, which supports our result of the combinational treatment on KEAP1 null GC cells. Taken together. PI3K and MAPK signaling may also play important roles in Lapatinib resistance in the HER2 amplified GC patients harboring loss of function mutations of NF1 or KEAP1. Merging our finding and previous studies, we draw a schematic diagram showing potential HER2-related signaling pathways and action mechanisms of various inhibitors in HER2 amplified GC (FIG. 7). Additional studies would be helpful to elucidate the molecular mechanisms of the drug resistance induced by these gene mutations.

The phase III randomized clinical trial with anti-HER2 monoclonal antibody, Trastuzumab, plus chemotherapy has been shown to improve median overall survival significantly in patients with HER2-positive gastric/gastro-esophageal junction cancer compared with chemotherapy alone⁹. Because of the promising positive results from the clinical trials with Lapatinib in HER2-positive breast cancer, several recent studies have been conducted to evaluate the efficacy of Lapatinib in GC. Two major clinical trials revealed that Lapatinib plus Paclitaxel demonstrated activity in the first of second-line treatment of patients with advanced gastric, gastroesophageal cancers but it did not significantly improve the prognosis even for the HER2-positive advanced GC patients^{10, 37}. Therefore, currently Lapatinib is not recommended for GC patients regardless of HER2 status. To identify indication or contraindication of this drug, among several clinicopathologic markers, age and ethnicity were addressed as potential markers for the effect of Lapatinib¹⁰. However, other than using these ambiguous “clinical markers”, it is important to identify more reliable biomarkers that can predict which patients will benefit from the treatment with the dual EGFR/HER2 inhibitor. Our study provides scientific evidence supporting the combinational usage of PI3K inhibitor and MEK inhibitor as a promising treatment option for HER2 positive GC who were resistant to Lapatinib or Trastuzumab.

In summary, CRISPR library screening provides a valuable platform for novel drug target discovery and validation. Our study has validated the approach, revealing the potential molecular mechanisms for the treatment of subsets of GC cases: loss-of-function mutation of CSK or PTEN causes resistance to Lapatinib in HER2 amplified OC cells via hyperactivation of PI3K and MAPK pathways, which can be overcome by applying drug combination of Lapatinib, PI3K and MAPK pathway inhibitors. The current study extends the understanding of Lapatinib resistance in HER2 amplified GC, which would facilitate to develop alternative treatment strategy to increase efficacy of anti-HER2 treatment.

### Materials and Methods

Cell Culture and Reagents

Human GC cell lines (N87, OE19) were obtained from the American Type Culture Collection (ATCC). All cell lines were cultured in RPMI1640 medium (Life Technologies) with 10% FBS (Life Technologies), penicillin (100 U/mL; Life Technologies), and streptomycin (100 U/mL; Life Technologies). All cells were maintained in a humidified incubator with 5% CO₂ at 37° C. Drug treatment reagents Lapatinib, Trastuzumab. LY294002, Saracatinib (AZD0530), Rapamycin, Trametinib (GSK1120212) and Copanlisib (BAY 80-6946) were purchased from Selleckchem.

CRISPR Library Gene Knockout Screening

The human GeCKO lentiviral pooled library lentiCRISPR v2 in one plasmid system was purchased from Addgene (Cat #1000000048) as two half-libraries (library A and library B). Genome-wide loss of function screen using GeCKO library was carried out as described e. Briefly, the library plasmid DNA was transformed using electroporation method in Lucigen Endura electrocompetent cells (Lucigen). The grown colonies were recovered from the plates, followed by plasmid DNA extraction using the Endotoxin-Free NucleoBond Xtra Maxi Plus EF kit (Takara). For lentiviral transduction, 293FT cells were co-transfected withlentiCRISPRv2 half-library A or B vector DNA, pCMV-VSVg and psPAX2 (Addgene) using Lipofectamine 2000 and PLUS reagent (ThermoFisher Scientific). After 48 h. supernatants from the transfected 293FT cells were harvested and concentrated using Lenti-X concentrator (Takara) according to the manufacturer's instructions. Pooled lentiviral libraries are transduced to 1×10⁸ GC cells with 3×10⁶ cells plated per transduction well. The multiplicity of infection (MOI) is about 0.3 to ensure that most cells receive only one stably integrated RNA guide. Puromycin (1.5 μg/mL for OE19 cells and 0.75 ug/ml for N87 cells) was added to the cells at 24 h post transduction and maintained for 7 days. Baseline cells were harvested after puromycin selection. Then transduced GC cells were treated with Lapatinib (1 μM for OE19 cells and 0.5 μM for N87 cells) or an equal volume DMSO for 14 days and the survived cells were harvested. For each cell line, two separate infection replicates were performed. The genomic DNA was extracted for PCR amplification and deep sequencing of the genomic regions containing the gRNAs was conducted. All deep sequencing data arc available at GEO.

Validation of Candidate Genes

For validation study, selected gRNAs that target the candidate genes were individually synthesized and cloned into the lentiCRISPR V2 plasmid (addgene, #52961). Viral particles were generated as described above. Then N87 and OE19 cells were infected with the corresponding viruses and the Lapatinib resistance was examined by treating the cells with indicated doses of Lapatinib for 6 days. Cell viability assay was performed as described below at the end of treatment.

Cell Viability Assay

For the cell viability assays, 4,000 cells/each well in a 96-well plate were treated with indicated drugs for 6 days and cell viabilities were measured using the CellTiter-Glo® luminescent cell viability assay kit according to the manufacturer's instructions (Promega). The luminescence intensity was measured using a multi-label plate reader (SpectraMax M5, Molecular Devices). The cell viabilities were calculated as relative values compared to the untreated controls.

Western Blotting

Cells were lysed with RIPA lysis buffer (Thermofisher Scientific) supplemented by protease inhibitor/phosphatase inhibitor cocktails (Cell signaling Technology). Lysates were separated on NuPAGE™ 4-12% Bis-Tris protein gels (Invitrogen) and were transferred to PVDF membranes (Millipore). The membranes were blocked with 5% fat-free milk (Cell signaling Technology) dissolved in TBST buffer (50 mM Tris-HCl, 150 mM NaCl, 0.1% Tween-20). Then, the membranes were incubated with primary antibodies overnight at 4° C. CSK antibody (#4980), PTEN antibody (#9188), MAPK1/2 antibody(#9102), Phospho-MAPK 1/2 (Thr202/Tyr204) antibody (#4370). Phospho-AKT (Ser473) antibody(#9271), AKT antibody (#9272) were purchased from Cell Signaling Technologies. GAPDH antibody (FL-335) was obtained from Santa Cruz biotechnology and horseradish peroxidase-conjugated secondary antibodies (anti-rabbit: NA934V, anti-mouse: NA931V) were purchased from GE healthcare. SuperSignal West Pico Chemiluminescent Substrate (Pierce) was used to detect signals.

Caspase-Glo 3/7 Apoptosis Assay

Caspase activity was detected by using Caspase-Glo 3/7 assay kit (Promega). Briefly. The GC cells were seeded in 96-well white luminometer assay plates at a density of 4,000 cells per well and incubated at 37° C. Cells were treated with Lapatinib for 48 h, 100 ul caspase 3/7 reagents were added to each well and incubated for 1 h on rotary shaker at room temperature. The luminescence intensity was measured using a multi-label plate reader (SpectraMax M5, Molecular Devices).

CRISPR Library Data Processing and Initial Analysis

Raw FASTQ files were trimmed using customized scripts. To align the processed reads to the library, the designed gRNA library sequences were assembled into a Burrows-Wheeler index using the Bowtie build-index furction³⁹. The qualities of fastq files are evaluated using fastqc with options “-Q33-q 25-p 50”. Then high quality reads are mapped to the screening library with <2 bp mismatches using Bowtie, and the raw read counts of gRNAs from all samples were merged into a count matrix. Next the effects of gene knockout were estimated using three different algorithms: Model-based Analysis of Genome-wide CRISPR-Cas9 Knockout (MAGeCK) Robust Rank Aggregation (RRA)⁴⁰, MAGeCK MLE⁴¹ and edgeR algorithms⁴². MAGeCK RRA algorithm builds a mean-variance model to estimate the variance of the read counts, and uses these variance estimations to model the read count changes for each gRNA in the treatment samples relative to the control samples. The read count changes (gRNA scores) of all gRNAs targeting each gene are then ranked and summarized into one score for the gene (gene score), using a modified RRA algorithm. MAGeCK-MLE initially use the raw table of reads as input, and models the read count of each gRNA for each sample by a negative binomial random variable and estimates the essentiality of genes in a CRISPR screen via a maximum likelihood approach. The edgeR algorithm uses high-throughtput sequencing counts to detect significantly selected gRNAs and genes by negative binomial method.

RNAseq and Data Analysis

Total RNA was extracted from indicated GC cell lines using RNeasy mini kit (QIAGEN®), mRNA-Seq libraries for the Illumina platform were generated and sequenced at NOVOGENE® (California. USA). For the RNAseq raw sequencing reads, we used HISAT2⁴³ to generate indexes and to map reads to the human genome build hg19. For assembly, we chose SAMtools⁴⁴ and the HTSeq⁴⁵ as the gene-level read counts could provide more flexibility in the differential expression analysis. Both HISAT2 and HTSeq analyses were conducted using the high performance research computing resources provided by Jackson Laboratory for Genomic Medicine in the Linux operating system. Differential expression and statistical analysis were performed using DESeq2 (release 3.7) in RStudio (version 1.1.447). We used variance stabilizing transformation to account for differences in sequencing depth. P-values were adjusted for multiple testing using the Benjamini-Hochberg procedure. A false discovery rate adjusted p-value <0.05 was set for the selection of DEGs, with differential expression defined as |log 2 (ratio)|≥0.585 (±1.5-fold) with the FDR set to 5%. Gene: were sorted according to their log 2-transformed fold-change values after shrinkage in DESeq2 and used for gene set enrichment analysis (GSEA)⁴⁶. Significant gene sets were used to perform the leading-edge analysis. We detected clusters of pathways that shared many leading-edge genes using a community detection algorithm and manually curated these clusters to elucidale the important phenotype-associated pathway groups visualized on the bar plots. Gene pathway analysis was conducted with the Kyoto Encyclopedia of Genes and Genomes (KEGG) collection of online databases dealing with genomes, enzymatic pathways, and biological chemicals⁴⁷.

Whole Exome Sequencing (WES) Data Analysis

For the 103 GC samples in previous publication²⁹, we downloaded the raw reads From European Nucleotide Archive with accession number PRJEB10531. For the variant analysis using Whole Exome sequencing data, all sequencing reads were submitted to a quality control check using FASTX-Toolkit (hannonlab.eshl.edu/fastx_toolkit/). The phred value 20 was chosen as the minimum threshold for base quality. Following is the alignment of resulting reads to hg19 reference genome with Burrows-Wheeler Aligner⁴⁸ and Picard (boadinstitute.github.io/picard/) was applied for post-alignment procedures as sorting, indexing, and marking duplicates. The alignments were submitted to local realignment around INDELs and base quality score recalibration by using the Genome Analysis Toolkit (GATK) version 3.5. Single nucleotide variants (SNVs) were identified using MuTect2 on the pre-processed sequencing data with default parameters. For Copy Number Variants (CNVs), the XHMM (eXone-Hidden Markov Model)⁴⁹ C++ software was run to detection CNVs from exome sequencing data. XHMM includes several key steps in running depth of coverage calculations, data normalization, CNV calling, and statistical genotyping and involves a number of parameters. In our study, we set all parameters to default (minTargetSize: 10; maxTargetSize: 10,000; minMeanTargetRD: 10; maxMeanTargetRD: 500; minMeanSampleRD: 25; maxMeanSampleRD: 200; maxSdSampleRD: 150) for filtering samples and targets, and prepared the data for normalization via XHMM.

### REFERENCES

1. Jemal A, Bray F, Center M M, Ferlay J, Ward E, Forman D Global cancer statistics. CA Cancer J Clin. 2011; 61(2):69-90. doi: 10.3322/caac.20107.
2. Sitarz R, Skierucha M. Mielko J. Offerhaus G J A, Maciejewski R, Polkowski W P. Gastric cancer: epidemiology, prevention, classification, and treatment. Cancer Manag Res, 2018; 10:239-248. doi: 10.2147/CMAR.S149619.
3. Siegel R L, Miller K D, Jemal A. Cancer statistics, 2017. CA Cancer J Clin. 2017:67(1):7-30. doi:10.3322/caac.21387.
4. Van Cutsem E, Bang Y-J, Peng-Yi F, et al. HER2 screening data from ToGA: targeting HER2 in gastric and gastroesophageal junction cancer. Gastric Cancer, 2015; 18(3):476-484. doi:10.1007/s10120-014-0402-y.
5. Ayed-Guerfali D, Abid N, Kbabir A, at al. Expression of human epidermal growth factor receptor 2 and p53 in gastric cancer patients: Clinical and prognosis relevance. Clip Cancer Investig J. 2016; 5(5):424. doi:10.4103/2278-05:3.197866.
6. Gravalos C, Jimeno A. HER2 in gastric cancer: a new prognostic factor and a novel therapeutic target. Ann Oncol. 2008; 19(9):1523-1529 doi:10.1093/annonc/mdn169.
7. Wong H. Yau T. Targeted Therapy in the Management of Advanced Gastric Cancer: Are We Making Progress in the Era of Personalized Medicine? Oncologist 2012:17(3):346-358. doi:10.1634/theoncologist.2011-0311.
8. Amir E, Ocaña A, Seruga B, Freedman O, Clemons M. Lapatinib and HER2 status: Results of a meta-analysis of randomized phase III trials in metastatic breast cancer. Cancer Treat Rev, 2010; 36(5):410-415. doi:10.1016/j.ctrv.2009.12.012.
9. Ban; Y-J, Van Cutsem E, Feyereislova A, et al. Trastuzumab in combination with chemotherapy versus chemotherapy alone for treatment of HER2-positive advanced gastric or gastro-oesophageal junction cancer (ToGA): a phase 3, open-label, randomised controlled trial. Lancet. 2010; 376(9742):687-697. doi:10.1016/S0140-6736(10):61121-X.
10. Hecht J R, Bang Y-J, Qin S K, et al. Lapatinib in Combination With Capecitabine Plus Oxaliplatin in Human Epidermal Growth Factor Receptor 2-Positive Advanced or Metastatic Gastric, Esophageal, or Gastroesophageal Adenocarcinoma: TRIO-013/LOGiC—A Randomized Phase III Trial. J Clin Oncol. 2016; 34(5):443-451. doi:10.1200/JCO.2015.62.6598.
11. Jarjigian Y Y. Lapatinib in gastric cancer. What is the LOGiCal next step? J Clin Oncol. 2016; 34(5):401-403. doi:10.1200/JCO.2015.64.2892.
12. Chia-Tung Chen1, Hyaehwan Kim1, David Liskal, Sizhi Gao2, James G Christensen3 A. Weiser M R. MET activation mediates resistance to lapatinib inhibition of HER2-amplified gastric cancer cells. Mol Cancer Ther. 2012; 11(3):660-669. doi:10.1016/j.ajo.2009.07.014.Aqueous.
13. Kim J, Fox C. Peng S, et al. Preexisting oncogenic events impact trastuzumab sensitivity in ERBB2-amplified gastroesophageal adenocarcinoma. J Clin Invest. 2014; 124(12):5145-5158 doi:10.1172/JCI75200.
14. Shalem O, Sanjana N E, Hartenian E, et al Genome—scale CRISPR—Cas9 knockout screening in human cells. Science (80-), 2014:343(6166):84-87. doi:10.1126/science.1247005.Genome-Scale.
15. Koike-Yusa H, Li Y, Tan E-P, Velasco-Herrera M D C, Yusa K. Genome-wide recessive genetic screening in mammalian cells with a lentiviral CRISPR-guide RNA library. Nat Biotechnol. 2014; 32(3):267-273. doi:1038/nbt.2800.
16. Evers B, Jastrzebski K, Heijmans J P M, Grernrum W, Beijersbergen R L, Bernards R. CRISPR knockout screening outperforms shRNA and CRISPRi in identifying essential genes. Nat Biotechnol. 2016; 34(6):631-633. doi:10.1038/nbt.3536
17. Okada M. Regulation of the SRC family kinases by Csk. Int J Bio Sci, 2012:8(10):1385-1397. doi:10.7150/ijbs.5141.
18. Szklarezyk D. Franceschini A. Wyder S, et al. STRING v10: protein-protein interaction networks, integrated over the tree of life. Nucleic Acids Res. 2015:43(D1):D447-D452. doi:10.1093/nar/gka1003.
19. Okines A F C, Cunningham D. Trastuzumab: a novel standard option for patients with HER-2-positive advanced gastric or gastro-oesophageal junction cancer. Therap Adv Gastroenterol, 2012; 5(5):301-318. doi:10.1177/1756283X12450246.
20. FDA News Alert: FDA approves new treatment for adults with relapsed follicular lymphoma. Food and Drug Administration. (Release date, Sep. 14, 2017) https:/www.fda.gov/newsevents/newsroom/pressannouncements/ucm576129.htm
21. Eroglu Z, Ribas A. Combination therapy with BRAF and MEK inhibitors for melanoma: latest evidence and place in therapy. Ther Adv Med Oncol. 2016; 8(1):48-56. doi:10.1177/1758834015616934.
22. Molecular targeted therapy: Treating cancer with specificity. Eur J Pharmacol. 2018:834:188-196. doi:10.1016/J.EJPHAR.2018.07.034.
23. Xia W, Mullin R. Keith B R, et al. Anti-tumor activity of GW572016: a dual tyrosine kinase inhibitor blocks EGF activation of EGFR/erbB2 and downstream Erk 1/2 and AKT pathways. Oncogene. 2002; 21(41):6255-6263. doi:10.1038/sj.one.1205794.
24. Biller J R, Elajaili H. Meyer V, Rosen G M, Eaton S S, Eaton G R. MET activation mediates resistance to lapatinib inhibition of HER2-amplified gastric cancer cells. Mol Cancer Ther. 2012; 236(3):47-55. doi:10.1016/j.jmr.2013.08.006.
25. Zhang Z. Wang J, Ji D, et al. Functional Genetic Approach identifies MET, HER3, IGF1R, INSR Pathways as Determinants of Lapatinib Unresponsiveness in HER2-Positive Gastric Cancer. Clin Cancer Res. 2014:20(17):4559-4573. doi-10.1158/1078-0432 CCR-13-3396.
26. Lu Y, Yu Q. Liu J H, et al. Src family protein-tyrosine kinases alter the function of PTEN to regulate phosphatidylinositol 3-kinase/AKT cascades. J Biol Chem. 2003; 278(41):40057-40066. doi:10.1074/jbc.M303621200.
27. Ebbesen S H, Scaltriti M. Bialucha C U, et al. Pten loss promotes MAPK pathway dependency in HER2/neu breast carcinomas. Proc Nord Acad Sci USA, 2016; 113(11):3030-3035. doi:10.1073/pnas.1523693113.
28. Cerami E. Gao J. Dogrusoz U, et al. The eBio Cancer Genomics Portal: An Open Platform for Exploring Multidimensional Cancer Genomics Data: FIG. 1. Cancer Discov, 2012; 2(5):401-404. doi:10.1158/2159-8290.CD-12-0095.
29. Park H, Cho S-Y, Kim H, et al. Genomic alterations in BCL2L1 and DLC1 contribute to drug sensitivity in gastric cancer. Proc Natl Acad Sci. 2015; 112(40):12492-12497. doi:10.1073/pnas.1507491112.
30. Cizkova M, Dujaric M-E. Lehmann-Che J, et al. Outcome impact of PIK3CA mutations in HER2-positive breast cancer patients treated with trastzumab. Br J Cancer. 2013:108(9):1807-1809. doi:10.1038/bjc.2013.164.
31. Eichhorn P J A, Gili M. Scaltriti M, et al. Phosphatidylinositol 3-kinase hyperactivation results in lapatinib resistance that is reversed by the mTOR/phosphatidylinositol 3-kinase inhibitor NVP-BEZ235. Cancer Res. 2008:68(22):9221-9230 doi:10.1158/0008-5472.CAN-08-1740.
32. de Bruin E C, Cowell C, Warne P H, et al. Reduced NF1 expression confers resistance to EGFR inhibition in lung cancer. Cancer Discov. 2014; 4(5):606-619. doi:10.1158/2159-8290.CD-13-0741.
33. Gibney G T, Smalley K S M An unholy alliance cooperation between BRAF and NF1 in melanoma development and BRAF inhibitor resistance. Cancer Discov. 2013:3(3):260-263. doi:10.1158/2159-8290.CD-13-0017.
34. Singh A, Misra V, Thimmulappa R K, et al. Dysfunctional KEAP1-NRF2 Interaction in Non-Small-Cell Lung Cancer. Meyerson M, ed. PLoS Med, 2006; 3(10):e420. doi:10.1371/journal.pmed.0030420.
35. Wang X-J, Sun Z, Villeneuve N F, et al. Nrf2 enhances resistance of cancer cells to chemotherapeutic drugs, the dark side of Nrf2. Carcinogenesis. 2008; 29(6):1235-1243. doi:10.1093/carcin/bgn095.
36. Nakaso K, Yano H, Fukuhara Y, Takeshima T, Wada-Isoe K, Nakashima K PI3K is a key molecule in the Nrf2-mediated regulation of antioxidative proteins by hemin in human neuroblastoma cells. FEBS Lett. 2003; 546(2-3):181-184. doi:10.1016/S0014-5793(03)00517-9.
37. Satoh T, Doi T, Ohtsu A, et al. Lapatinib plus paclitaxel versus paclitaxel alone in the second-line treatment of HER2-amplified advanced gastric cancer in Asian populations: TyTAN—A randomized, phase III study. J Clin Oncol. 2014; 32(19):2039-2049. doi:10.1200/JCO.2013.53.6136.
38. Joung J, Konermann S, Gootenberg J S. Protocol: Genome-scale CRISPR-Cas9 Knockout and Transcriptional Activation Screening, 2016.
39. Langmead B, Trapnell C, Pop M, Salzberg S L, Ultrafast and memory-efficient alignment of short DNA sequences to the human genome. Genome Biol. 2009; 10(3):R25. doi:10.1186/gb-2009-10-3-r25.
40. Li W, Xu H, Xao T, et al MAGeCK enables robust identification of essential genes from genome-scale CRISPR/Cas9 knockout screens. Genome Biol. 2014; 15(12):554. doi:10.1186/s13059-014-0554-4.
41. Li W, Köster J. Xu H, et al. Quality control, modeling, and visualization of CRISPR screens with MAGeCK-VISPR Genome Biol. 2015; 16:281. doi:10.1186/s13059-015-0843-6.
42. Roninson M D, McCarthy D J, Smyth G K edgeR: s Bioconductor package for differential expression analysis of digital gene expression data. Bioinformatics, 2010; 26(1):139-140. doi:10.1093/bioinfomatics/brp616.
43. Kim D, Langmead B. Salzberg S L. HISAT: a fast spliced aligner with low memory requirements. Nat Methods. 2015; 12(4):357-360. doi:10.1038/nmeth.3317.
44. Li H. Handsaker B. Wysoker A, et al. The Sequence Alignment/Map format and SAMtools. Bioinformatics, 2009; 25(16):2078-2079. doi:10.1093/bioinformatics/btp352.
45. Anders S, Pyl P T, Huber W, HTSeq—a Python framework to work with high-throughput sequencing data. Bioinformatics, 2015; 31(2):166.169. doi:10.1093/bioinformatics/btu638.
46. Subramanian A, Tamayo P, Moontha V K, et al. Gene set enrichment analysis: a knowledge-based approach for interpreting genome-wide expression profiles. Proc Natl Acad Sci USA. 2005; 102(43):15545-15550. doi:10.1073/pnas.0506580102.
47. Kanehisa M, Furumichi M, Tanabe M, Sato Y, Morishima K. KEGG: new perspectives on genomes, pathways, diseases and drugs. Nucleic Acid Res. 2017:45(D1):D353-D361. doi:10.1093/nar/gkw1092.
48. Li H. Durbin R. Fast and accurate short read alignment with Burrows-Wheeler transform. Bioinformatics, 2009; 25(14):1754-1760. doi:10.1093/bioinformatics/fbtp324.
49. Discovery and Statistical Genotyping of Copy-Number Variation from Whole-Exome Sequencing Depth. Am J Hum Genet, 2012; 91(4):597-607. doi:10.1016/J.AJHG.2012.08.005.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising.” “including,” “carrying,” “having.” “containing.” “involving,” “holding,” “composed of,” and the like arc to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures. Section 2111.03.

The terms “about” and “substantially” preceding a numerical value mean±10% of the recited numerical value.

Where a range of values is provided, each value between the upper and lower ends of the range are specifically contemplated and described herein.

1. A method of treating gastric cancer in a subject, the method comprising:
(a) administering to the subject lapatinib; and
(b) administering to the subject a phosphoinositide 3-kinase (PI3K) inhibitor, a MEK inhibitor, or a combination of a PI3K inhibitor and a MEK inhibitor.

2. The method of claim 1, wherein the gastric cancer is HER2-amplified gastric cancer.

3. The method of claim 1, wherein step (b) comprises administering to the subject a PI3K inhibitor and a MEK inhibitor.

4. The method of claim 1 wherein the PI3K inhibitor comprises copanlisib.

5. The method of claim 1, wherein the MEK inhibitor comprises trametinib.

6. The method of claim 1, wherein the lapatinib and the PI3K inhibitor, the lapatinib and the MEK inhibitor, or the lapatinib, the PI3K inhibitor, and the MEK inhibitor are administered simultaneously.

7. The method of claim 1, wherein the ratio of lapatinib to PI3K inhibitor is 1:2, the ratio of lapatinib to MEK inhibitor is 1:2, and the ratio of PI3K inhibitor to MEK inhibitor is 1:1.

8. The method of claim 1, wherein the lapatinib, the PI3K inhibitor, or the MEK inhibitor is administered intravenously or orally.

9. The method of claim 1, wherein the gastric cancer cells do not express, or express a reduced level of, a CSK gene and/or a PTEN gene.

10. A method, comprising:
(a) contacting gastric cancer cells with lapatinib; and
(b) contacting the gastric cancer cells with a PI3K pathway inhibitor, a MAPK pathway inhibitor, a SRC family inhibitor, an mTOR inhibitor, or a combination thereof.

11. The method of claim 10, wherein the gastric cancer cells are HER2-amplified gastric cancer cells.

12. The method of claim 10, wherein the SRC family inhibitor comprises saracatinib.

13. The method of claim 1, wherein the mTOR inhibitor comprises rapamycin.

14. The method of claim 1, wherein the PI3K pathway inhibitor comprises a PI3K inhibitor.

15. The method of claim 14, wherein the PI3K inhibitor comprises copanlisib and/or LY294002.

16. The method of claim 1, wherein the MAPK pathway inhibitor comprises a MEK inhibitor.

17. The method of claim 16, wherein the MEK inhibitor comprises trametinib.

18. The method of claim 1, wherein the gastric cancer cells do not express, or express a reduced level of, a gene selected from CSK, PTEN, BAX, KCTD5, KEAP1, NF1, and TADA1.

19. A kit comprising:
(a) lapatinib; and
(b) a PI3K pathway inhibitor, a MAPK pathway inhibitor, or a PI3K pathway inhibitor and a MAPK pathway inhibitor.

20.-24. (canceled)

25. A method, comprising:
(a) delivering in vitro to control cells and to human gastric cancer cells harboring HER2 amplification a pooled genome-scale CRISPR-Cas9 knockout library;
(b) treating the controls cells and the human gastric cancer cells of step (a) with lapatinib;
(c) extracting DNA from the lapatinib-treated controls cells and the lapatinib-treated human gastric cancer cells of step (b);
(d) sequencing the DNA extracted from step (c); and
(e) identifying from the sequenced DNA of step (d) candidate loss-of-function genes that may contribute to lapatinib resistance.

26.-30. (canceled)
